(19) United States
(12) Patent Application Publication
(10) Pub. No.: US 2024/0009234 A1
(43) Pub. Date: Jan. 11, 2024

(54) SYNTHETIC OLIGOMERIZATION SYSTEMS FOR CELL ENGINEERING AND THERAPY

(71) Applicant: The Board of Trustees of the Leland Stanford Junior University, Stanford, CA (US)

(72) Inventors: Lei S. Qi, Stanford, CA (US); Michael Chavez, Stanford, CA (US); Paul B. Finn, Stanford, CA (US)

(73) Assignee: The Board of Trustees of the Leland Stanford Junior University, Stanford, CA (US)

(21) Appl. No.: 18/007,283

(22) PCT Filed: Jul. 28, 2021
(86) PCT No.: PCT/US2021/043563

§ 371 Date: Jan. 27, 2023

Related U.S. Application Data
(60) Provisional application No. 63/058,466, filed on Jul. 29, 2020.

(51) Int. Cl. A61K 35/17; C12N 5/078; C07K 14/715; C07K 14/71; A61K 39/00

(52) U.S. Cl. CPC A61K 35/17; C12N 5/0634; C07K 14/7155; C07K 14/71; A61K 39/4631; A61K 39/464419; A61K 39/464431; A61K 2239/21; A61K 2239/22; A61K 2239/17; C07K 2317/569; C07K 2317/622

(57) ABSTRACT

Provided herein are chimeric transmembrane receptor polypeptides configured to oligomerize upon recognition of an extramembrane signal. The receptors include an extramembrane domain, a transmembrane domain, and an intramembrane domain configured to induce activation of one or more intramembrane signal pathways upon oligomerization of the receptor. The provided receptors are particularly useful for engineered cell therapies. Also provided are systems and host cells including the disclosed receptors, and methods for using the disclosed materials.

### CROSS-REFERENCE TO RELATED APPLICATIONS

The present application claims priority to U.S. Provisional Application No. 63/058,466 filed Jul. 29, 2020, the full disclosure of which is incorporated by reference in its entirety for all purposes.

### BACKGROUND

Small molecule therapeutics and biologics have dominated the pharmaceutical space in the last century. However, the novel paradigm of cell therapy is beginning to change the pharmaceutical landscape and revolutionize the treatment of diseases. By utilizing cells as a therapy, the human body's natural and complex functionality can be reprogrammed towards new targets, opening an enormous space of possible therapeutic modalities that were previously unavailable using old paradigms. For example, in the adoptive cell therapy of cancer treatment, immune cells are extracted from the patient, engineered with novel functionality through molecular addition or genetic modification, and reintroduced to the patient to eliminate cancer cells. A successful clinical example is CAR T cell therapy, a novel way to treat liquid cancers in patients who have relapsed or become refractory to traditional forms of therapy such as radiation and chemotherapy. In this therapy, T cells are engineered with a chimeric antigen receptor (CAR) that imbues the T cells with the ability to target cancer and subsequently activate native T cell functionality to target and kill the malignancy. By redirecting T cells toward a new cancer cell target, cells can survey their entire host system and mount immune responses against cancer cells, resulting in remission rates that were previously unobtainable with other forms of therapy.

The success of engineered cell therapies benefits from such rewiring of natural immune activation pathways through genetic manipulation to attack novel disease-relevant targets. Many challenges and unfilled niches exist within this space, though. First, current successful cell therapies solely rely on the CAR or T cell receptor (TCR) engineered into T cells. The efficacy of CAR or TCR T cells towards most solid tumor by using this approach is very limited. In addition, there has been limited success using other molecular compositions. Defining novel molecular compositions that imbue new features to immune cells is therefore a key aspect for innovation needed to bring cell therapy to unmet indications.

Second, engineered immune cell activation is currently only controlled by disease signals. In this way, the cells act autonomously and outside the control of a physician or through a secondary signal. This unchecked autonomous activity can often lead to cytokine release syndrome or other severe side effects on healthy organs. Additionally, therapies can also suffer from uncontrollable variation from patient to patient.

Third, the vast majority of previously available synthetic tools activate only one specific signaling pathway in engineered T cells with limited efficacy. In contrast, multiple signaling pathways in a natural T cell give rise to differential signaling dynamics and, therefore different immune responses. This incapability to modulate diverse signaling pathways within engineered T cells poses another major challenge to the application of these immune cells to treat solid tumors or autoimmune diseases.

Finally, T cells are adaptive immune cells, comprising a small portion of the overall immune system. There are no currently available tools to engineer or utilize other innate immune cells such as macrophages, dendritic cells, and neutrophils to treat disease. Novel molecular tools that can activate multiple pathways in these innate immune cells could allow cell therapies to expand into new areas such as infectious diseases (both viral and bacterial), wound healing, aging, and autoimmunity.

In view of these observations and results, there is a need in the art for new systems for engineering cells that are useful in improved therapies.

### BRIEF SUMMARY

The disclosure herein provides a set of solutions to address these challenges and provide associated and other advantages. These include molecular designs and compositions, and methodologies of utilizing such systems for better cell therapies. The provided materials and methods have broad applicability with both adaptive and innate immune cells, and enable clinical features that can impact broad disease indications.

In one aspect, the disclosure provides a chimeric transmembrane receptor polypeptide configured to oligomerize upon recognition of an extramembrane signal by the chimeric transmembrane receptor polypeptide. The chimeric transmembrane receptor polypeptide includes an extramembrane domain, a transmembrane domain, and an intramembrane domain. The intramembrane domain is configured to induce activation of one or more intramembrane signal pathways upon oligomerization of the chimeric transmembrane receptor polypeptide. In some embodiments, the chimeric transmembrane receptor polypeptide is configured to dimerize upon recognition of the extramembrane signal, wherein the intramembrane domain is configured to induce activation of the one or more intramembrane signal pathways upon dimerization of the chimeric transmembrane receptor polypeptide.

In some embodiments, the extramembrane domain includes an FK506 binding protein (FKBP) family domain, a bromodomain and extra terminal domain (BET) family domain, a gibberellin-insensitive dwarf (GID) family domain, a B-cell lymphoma 2 (Bcl-2) family domain, or a variant or fragment thereof. In some embodiments, the extramembrane domain includes a soluble N-ethylmaleimide-sensitive factor (NSF) attachment protein (SNAP) receptor domain, a biotin receptor domain, an epidermal growth factor receptor domain, an estrogen receptor domain, an androgen receptor domain, an insulin receptor domain, a programmed cell death protein-1 (PD-1) domain, an AXL receptor tyrosine kinase domain, a single-chain variable fragment (scFv) domain, a nanobody domain, or a variant or fragment thereof. In some embodiments, the extramembrane domain includes a toll-like receptor (TLR) family domain, an ErbB receptor family domain, a type I cytokine receptor family domain, a type II cytokine receptor family domain, a transforming growth factor beta (TGFβ) receptor family domain, a tumor necrosis factor (TNF) receptor family domain, an immunoglobulin superfamily (IgSF) domain, a tropomyosin receptor kinase (trk) family domain, a glial cell-derived neurotrophic factor (GDNF) receptor family domain, or a variant or fragment thereof. In some embodiments, the chimeric transmembrane receptor polypeptide includes one or more additional extramembrane domains.

In some embodiments, the transmembrane domain includes a TLR family domain, an ErbB receptor family domain, a type I cytokine receptor family domain, a type II cytokine receptor family domain, a TGFβ receptor family domain, a TNF receptor family domain, an IgSF domain, a trk family domain, a GDNF receptor family domain, or a variant or fragment thereof. In some embodiments, the intramembrane domain includes a TLR family domain, an ErbB receptor family domain, a type I cytokine receptor family domain, a type II cytokine receptor family domain, a TGFβ receptor family domain, a TNF receptor family domain, an IgSF domain, a trk family domain, a GDNF receptor family domain, a gene editing nuclease domain, a transcriptional controller domain, an RNA controller domain, a protein controller domain, or a variant or fragment thereof, In some embodiments, the chimeric transmembrane receptor polypeptide includes one or more additional intramembrane domains.

In some embodiments, the chimeric transmembrane receptor polypeptide further includes a signal peptide. In some embodiments, the signal peptide includes a TLR family signal peptide, a CD3ε signal peptide, a CD8α signal peptide, an IgK signal peptide, an IgL signal peptide, a mouse CD4 signal peptide, or a variant or fragment thereof. In some embodiments, the chimeric transmembrane receptor polypeptide further includes one or more linker peptide sequences. In some embodiments, the one or more linker peptide sequences include a GGS linker sequence, a GGSGGSGGS linker sequence, a GS linker sequence, a GSGSGS linker sequence, an ESKYGPPAPPAP (mutant IgG4 hinge) linker sequence, an ESKYGPPCPPCP (IgG4 hinge), or a combination thereof.

In some embodiments, the extramembrane signal includes a ligand capable of binding to the extramembrane domain. In some embodiments, the ligand includes a small molecule. In some embodiments, the ligand includes an oligonucleotide. In some embodiments, the ligand includes one or more of a peptide, a protein, a polysaccharide, or a lipid. In some embodiments, the ligand includes one or more of an antibody, a nanobody, or an scfv. In some embodiments, the ligand includes a metabolite.

In some embodiments, the extramembrane signal includes a change in temperature or pH. In some embodiments, the extramembrane signal includes a change in sound or electromagnetic radiation. In some embodiments, the extramembrane signal includes a change in mechanical force.

In some embodiments, at least one of the one or more intramembrane signal pathways is an exogenous pathway. In some embodiments, at least one of the one or more intramembrane signal pathways is a synthetic pathway. In some embodiments, the one or more intramembrane signal pathways include pathways for one or more of genome sequence editing, transcription activation or repression, epigenetic modifications, genome translocation and rearrangement, RNA expression or degradation, RNA splicing or processing, post-transcription modifications of mRNA or mRNA, post-translational modifications of proteins, cleavage or proteolysis of proteins, production or degradation of metabolites or other chemistries, trafficking of signaling molecules, cell cycle control, cell differentiation or reprogramming, T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, or production of oncolytic virus.

In another aspect, the disclosure provides a system including a membrane separating an extramembrane region from an intramembrane region, and a chimeric transmembrane receptor polypeptide as disclosed herein. The extramembrane domain is located within the extramembrane region, and the intramembrane domain is located within the intramembrane region. In some embodiments, the membrane is a cellular membrane, a nuclear membrane, an organelle membrane, or a vesicle membrane. In some embodiments, the system further includes a trans-acting receptor polypeptide. In some embodiments, the trans-acting receptor polypeptide is a chimeric antigen receptor (CAR), a T cell antigen receptor (TCR), a Synthetic Notch (SynNotch) receptor, a GPCR TANGO receptor, a CRISPR ChaCha receptor, a B cell receptor (BCR), C-type lectin-like receptor Ly49, a CD94-NKG2C/E/H heterodimeric receptor, an NKG2D receptor, a DNAM-1/CD226 nectin/nectin-like binding receptor, a CRTAM nectin/nectin-like binding receptor, a member of the natural cytotoxicity receptor (NCR) family, a CD64 Fc receptor, a CD32 Fc receptor, a CD16a Fc receptor, a CD16b Fc receptor, a CD23 Fc receptor, a CD89 Fc receptor, a CD351 Fc receptor, an FcεRI Fc receptor, or an FcRn Fc receptor.

In another aspect, the disclosure provides a host cell including a chimeric transmembrane receptor polypeptide as disclosed herein. In some embodiments, the host cell expresses the chimeric transmembrane receptor polypeptide. In some embodiments, the host cell is a lymphocyte, a phagocytic cell, a granulocytic cell, or a dendritic cell (DC), e.g., a cDC1, a cDC2, a pDC, a tDC, or a monocyte-derived DC. In some embodiments, the lymphocyte is a T cell, a B cell, a natural killer (NK) cell, or an innate lymphoid cell (ILC), e.g., a Group 1 ILC, a Group 2 ILC, or a Group 3 ILC. In some embodiments, the T cell is a CD4 helper αβT cell, a CD8+ killer αβT cell, a δγT cell, or a natural killer T (NKT) cell, e.g., an invariant natural killer T (iNKT) cell. In some embodiments, the B cell is a plasma cell. In some embodiments, the phagocytic cell is a macrophage or a monocyte, e.g., a CD14+ monocyte or a CD16+ monocyte. In some embodiments, the granulocytic cell is a neutrophil, a basophil, an eosinophil, or a mast cell. In some embodiments, the host cell is a stem cell or a progenitor cell. In some embodiments, the host cell is an induced pluripotent stem cell (iPSC), an embryonic stem cell (ESC), an adult stem cell, or a mesenchymal stem cell (MSC). In some embodiments, the progenitor cell is a neural progenitor cell, a skeletal progenitor cell, a muscle progenitor cell, a fat progenitor cell, a heart progenitor cell, a chondrocyte, or a pancreatic progenitor cell

In another aspect, the disclosure provides a population of host cells. Each cell of the population independently includes a chimeric transmembrane receptor polypeptide as disclosed herein, or a system as disclosed herein.

In another aspect, the disclosure provides a method for activating an intramembrane signal pathway. The method includes providing a chimeric transmembrane receptor polypeptide as disclosed herein, a system as disclosed herein, a host cell as disclosed herein, or a population of host cells as disclosed herein. In some embodiments, the method further includes exposing the chimeric transmembrane receptor to the extramembrane signal.

In another aspect, the disclosure provides a method for preventing or treating a disease in a subject. The method includes administering to the subject an amount of a chimeric transmembrane receptor polypeptide as disclosed herein, a system as disclosed herein, a host cell as disclosed herein, or a population of host cells as disclosed herein. The amount administered to the subject is an amount therapeutically effective to prevent or treat the disease.

In some embodiments, the method further includes, subsequent to the administering, exposing the chimeric transmembrane receptor to the extramembrane signal. In some embodiments, the exposing includes introducing to the subject a therapeutically effective amount of the extramembrane signal. In some embodiments, the disease is a cancer. In some embodiments, the disease is a cancerous tumor. In some embodiments, the cancerous tumor is a solid cancerous tumor. In some embodiments, the cancerous tumor is a liquid cancerous tumor. In some embodiments, the disease is an infectious disease. In some embodiments, the infectious disease is a viral infectious disease or a bacterial infectious disease. In some embodiments, the disease is an autoimmune disease. In some embodiments, the disease is an age-related disease.

In another aspect, the disclosure provides a method for healing a wound in a subject. The method includes administering to the subject an amount of a chimeric transmembrane receptor polypeptide as disclosed herein, a system as disclosed herein, a host cell as disclosed herein, or a population of host cells as disclosed herein. The amount administered to the subject is an amount therapeutically effective to prevent or treat the disease. In some embodiments, the method further includes, subsequent to the administering, exposing the chimeric transmembrane receptor to the extramembrane signal. In some embodiments, the exposing includes introducing to the subject a therapeutically effective amount of the extramembrane signal.

### BRIEF DESCRIPTION OF THE DRAWINGS

FIG. 1 is an illustration showing a general design of the architecture of a provided chimeric transmembrane receptor polypeptide.

FIG. 2 is a graph showing the performance of a chimeric transmembrane receptor polypeptide composed of an FKBP extracellular domain and a TLR5 transmembrane and intracellular domain, where the chimeric transmembrane receptor polypeptide is in Jurkat cells and is activated by AP20187.

FIG. 3 is a graph showing successive truncations of natural TLR5 followed by N-terminal fusion of FKBP and activation by AP20187.NT refers to no treatment.

FIG. 4 is a graph showing loss of flagellin activation of truncated FKBP-TLR5 receptors.

FIG. 5 is a graph comparing activation of endogenous and engineered receptors by flagellin and AP20187.

FIG. 6 is a graph of a dose response curve for FKBP-TLR5 chimeric transmembrane receptor polypeptides activated by AP20187.

FIG. 7 is a graph showing activation of a two-receptor heterodimerization system composed of one chimeric transmembrane receptor polypeptide with an FKBP extracellular domain and a TLR5 transmembrane and intracellular domain, and a second chimeric transmembrane receptor polypeptide with an FRB extracellular domain and a TLR5 transmembrane and intracellular domain. The heterodimerization system is activated by AP21967.

FIG. 8 is a graph showing activation of a chimeric transmembrane receptor polypeptide composed of a caffeine dimerizable nanobody extracellular domain fused to a TLR5 transmembrane and intracellular domain, where the chimeric transmembrane receptor polypeptide is activated by caffeine.

FIG. 9 is a graph showing activation of TLR5 chimeric transmembrane receptor polypeptides containing different signal peptides.

FIG. 10 is a graph showing activation of an Mcl-TLR5 chimeric transmembrane receptor polypeptide by small molecule compounds that each contain the same Mcl-1 binding motif but that differ by linker length.

FIG. 11 is a graph showing the performance of a chimeric transmembrane receptor polypeptide composed of an FKBP extracellular domain and a TLR4 transmembrane and intracellular domain, where the chimeric transmembrane receptor polypeptide is in Jurkat cells and is activated by AP20187.

FIG. 12 is a graph showing activation of a chimeric transmembrane receptor polypeptide composed of an FKBP extracellular domain, a TLR5 transmembrane domain, and a TLR4 intracellular domain, where the chimeric transmembrane receptor polypeptide is activated by AP20187.

FIG. 13 is a graph showing activation of a chimeric transmembrane receptor polypeptide composed of an FKBP extracellular domain, a TLR5 transmembrane domain, and an IL6RST (gp130) intracellular domain, where the chimeric transmembrane receptor polypeptide is activated by AP20187.

FIG. 14 is a graph showing activation of a chimeric transmembrane receptor polypeptide composed of an epidermal growth factor receptor (EGFR) extracellular domain fused to a TLR5 transmembrane and intracellular domain, where the chimeric transmembrane receptor polypeptide is activated by the protein/peptide, epidermal growth factor (EGF).

FIG. 15 is a graph showing oligomerization-induced activation of a chimeric transmembrane receptor polypeptide composed of a monomeric streptavidin extracellular domain, a CD28 transmembrane domain, and an CD28-CD3ζ intracellular domain, where the chimeric transmembrane receptor polypeptide is in Jurkat cells and is activated by a multivalent inducer that can control dimer-, trimer-, or tetramerization.

FIG. 16 is a graph showing activation of a post-translationally oligonucleotide-modified chimeric transmembrane receptor polypeptide composed of a SNAP protein extracellular domain, a CD28 transmembrane domain, and CD28-CD3ζ intracellular domains, where the chimeric transmembrane receptor polypeptide is activated by a trimeric oligonucleotide.

FIG. 17 is a graph showing activation of the T cell signaling pathway through the NFAT transcription factor by oligomerization of a chimeric transmembrane receptor polypeptide composed of a monomeric streptavidin extracellular domain, a CD28 transmembrane domain, and CD28-CD3ζ intracellular domains.

FIG. 18 is a graph showing activation of the T cell signaling pathway through the NFκβ transcription factor by oligomerization of a chimeric transmembrane receptor polypeptide composed of a monomeric streptavidin extracellular domain, a CD28 transmembrane domain, and CD28-CD3ζ intracellular domains.

FIG. 19 is a graph showing activation of a chimeric transmembrane receptor polypeptide composed of a monomeric streptavidin extracellular domain, a CD28 transmembrane domain, and CD28-CD3ζ intracellular domains, where the chimeric transmembrane polypeptide is activated by a branched, multivalent small molecule activator.

FIG. 20 is a graph showing activation of a chimeric transmembrane receptor polypeptide composed of a monomeric streptavidin extracellular domain, a CD28 transmembrane domain, and a CD137 (4-1BB) intracellular domain.

FIG. 21 is a graph showing that the activated FKBP-TLR5 chimeric transmembrane receptor polypeptide can improve the ability of Natural Killer cells (NK-92 cell line) to kill MHC^{- cells (K}562 shown). Y-axis is a proxy for K562 cell number measured by live cell imaging.

### DETAILED DESCRIPTION

The inventors have designed a technology platform based on a synthetic transmembrane receptor that can transmit an extramembrane signal input across, for example, a cell or organelle membrane, to activate intramembrane signal pathways via customized ligand-mediated oligomerization. Advantageously, and unlike previously available engineered receptors that integrate extracellular signals by binding a membrane-bound antigen and transmitting that signal across the membrane through mechanical force, the provided chimeric transmembrane receptor polypeptide activates via oligomerization and not via mechanical signal transduction. This enables a novel suite of pathways, activators, and signals, such as soluble small molecules, to be associated with the provided materials and methods.

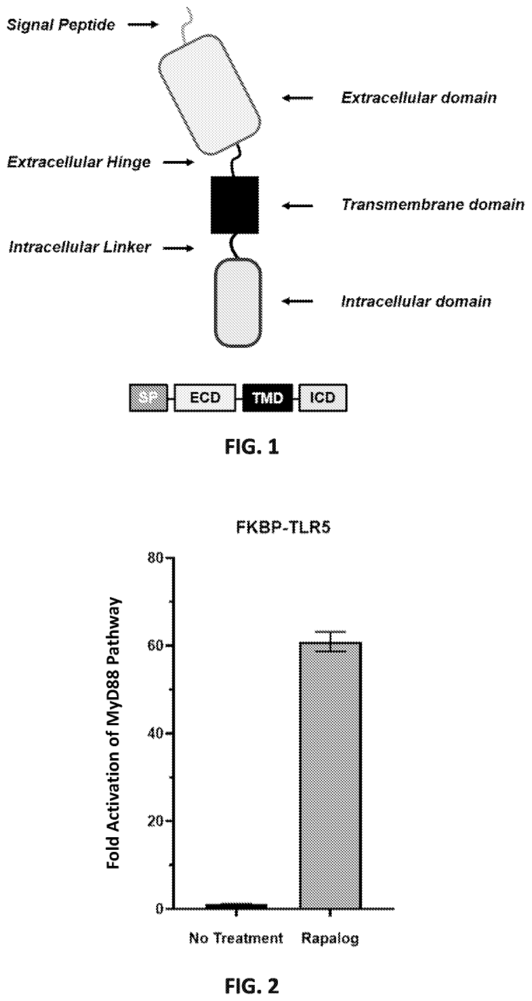

As shown in FIG. 1, the provided chimeric transmembrane receptor polypeptide can include: 1) an extramembrane, e.g., extracellular, domain: 2) a transmembrane domain: 3) an intramembrane, e.g., intracellular, domain: and 4) optional post-translational processing features, e.g., signal peptides that direct localization of the receptor, and linker peptide sequences that connect various domains. The general structure of the receptor polypeptide can be, for example, SP-ECD-TMD-ICD, where SP represents one or more signal peptides, ECD represents an extramembrane or extracellular domain, TMD represents a transmembrane domain, ICD represents an intramembrane or intracellular domain, and each dash (-) represents either a direct fusion of adjacent domains or a fusion with the addition of linkers The extracellular domain can oligomerize upon detecting an extramembrane signal, e.g., through ligand binding, which can pull intracellular domains together to activate, for example, downstream signals for signal pathway control, genome control, or cell function control.

Among the many benefits provided by the chimeric transmembrane receptor polypeptide of the systems and methods disclosed herein are that the receptor can be inducibly activate by the presence or absence of certain signals that can be either endogenous or exogenous. This induction can be reversed by removal of the signal or addition of competing signal to restore the receptor status or a host cell status to its original state. In addition, depending on the concentration or intensity of the signal, the receptor system can be controlled precisely by the amount of added signals to fine-tune its activity.

The chimeric transmembrane receptor polypeptide further allows the repurposing of many available small molecule drugs or antibodies as safe and effective inducers. This includes those drugs that are approved by FDA or those that failed merely due to compromised bioavailability and efficacy. The receptor system does not require that the signal enters host cells, so small molecules or antibodies that cannot enter cells can also be used as inducers. This greatly expands the compatibility of diverse safe molecules with the provided methods. Moreover, multiple receptors with various inputs and outputs can be combined with each other or combined with other T cell- or B cell-specific receptors to generate combinatorial functions. Importantly, when and where such receptor engineered cells are activated can be controlled by the location and administration of the extramembrane signal.

The provided materials and methods thus provide several advantages and improvements over engineered cell therapies previously available. For example, unlike first-generation CAR T cell therapeutic receptors which consist of an extracellular cancer binding domain and an intracellular T cell activating domain, and which imbue weak stimulation to T cells and display poor efficacy in killing cancer, the receptor disclosed herein provides a secondary signal to better control a CAR T cell response upon target engagement through the activation of synergistic signaling pathways to aid in tumor clearance and disease elimination.

Second-generation CAR T cell therapeutic receptors include both a costimulatory domain and a T cell activation domain on their intracellular side. The costimulatory domain improves the therapeutic response of T cells by increasing T cell proliferation or cytotoxicity. However, there is no control of the therapeutic window of these engineered cells, and significant side-effects can be created in patients. Because the costimulatory domain is tied to the CAR, these second-generation cells have a binary “all or nothing” response resulting only in full activity when binding to a cancer. In contrast, the provided chimeric transmembrane receptor polypeptide can afford control over the therapeutic window of an engineered cell, e.g., an engineered T cell. This can better allow a healthcare professional to both maintain efficacy and increase patient safety during a more tunable treatment.

T cell receptor (TCR)-engineered T cells can provide a cancer patient with a TCR that binds a particular marker indicative of a disease such as a specific type of cancer. TCR-engineered T cells genetically introduce the specific TCR into a patient's T cells, which are then reinfused into the patient to search for and destroy the targeted cancer. However, as with first-generation CARs, the cancer-binding signal insufficiently induces activation of the T cells. The provided chimeric transmembrane receptor polypeptide can be introduced to the patient's T cells along with the TCR to provide an important secondary signal that can allow the TCR therapy to work more effectively.

In START CAR T cells the extracellular binding domain and the intracellular costimulatory and activation domain are naturally separate, but a physician-given small molecule allows these separate pieces to dimerize and the T cell to perform its reprogrammed function. In the STOP CAR case, the extracellular and intracellular pieces are naturally dimerized, but a physician-given small molecule decouples the pieces to turn the CAR T cell off. In both cases, the small molecule drug must be cell permeable and the T cells have an “all or nothing” response. In the provided materials and methods, however, the small molecule activator can be cell impermeable as the receptor can be engineered to be at the cell surface, significantly increasing the variety of molecules that can be used as an extramembrane signal. In addition, the provided receptor, when used in conjunction with a CAR, can titrate the CAR response between the low response of a first-generation CAR and the high response of a second-generation CAR. In doing so, the cells can always have some level of activity, ensuring that cancer fighting capabilities are always present and that a therapy is not cleared from the patient system due to lack of stimulation.

Another engineered receptor, iMC, can be used in conjunction with a first-generation CAR and can be dimerized with a small molecule to act as the secondary signal for CAR T cell killing of cancer. The residence of iMC in the cytoplasm, though, requires that its small molecule inducer be cell permeable, greatly reducing the space of drugs that can activate it. Also, the iMC receptor was designed to function with a specific, and clinically unproven, costimulatory domain. Beneficially, the provided chimeric transmembrane receptor polypeptide is adaptable to many different costimulatory domains, including ones with FDA clearance and ones yet to be discovered.

The synNotch receptor contains an extracellular cell surface binding domain and an intracellular orthogonal transcription factor, and is agnostic to cell-type and output. The receptor must, however, recognize surface-bound antigens to function. The provided chimeric transmembrane receptor polypeptide can advantageously also recognize, for example, soluble factors created by the patient's body or administered as a drug. Furthermore, while the synNotch receptor output can be designed by genetically introducing a cellular program, the provided receptor can be connected to a multitude of natural pathways that activate various complex immune phenotypes.

The first of two parts of the MESA engineered receptor includes an extracellular binding domain connected to a transmembrane domain and an intracellular protease. The second part of MESA includes the same extracellular binding domain connected to a transmembrane domain and an intracellular activation domain connected to the protease cleavage site. In this way, a signal forces oligomerization of the two parts, bringing the protease and the protease cleavage site in proximity, and inducing cutting of the activation domain from the receptor. The thus freed activation domain is then able to move about the cell and activate pathways. The activation domains of MESA are therefore limited to a small subset of transcription factors and other activators that can be controlled by cellular localization (such as nuclear exclusion). Unlike the provided chimeric transmembrane receptor, MESA cannot be used to activate the multitude of cell signaling responses naturally induced by oligomerization. In addition, the MESA receptor is a heterodimer requiring two genetic components for expression whereas the provided receptor requires only one polypeptide.

### Transmembrane Receptor Polypeptides

In one aspect, a chimeric transmembrane receptor polypeptide is disclosed. The provided chimeric transmembrane receptor polypeptide is configured to oligomerize upon recognition of an extramembrane signal. In some embodiments, the receptor polypeptide is configured to form a dimer, e.g., a homodimer or heterodimer, upon recognition of the extramembrane signal. In some embodiments, the receptor polypeptide is configured to form a trimer, e.g., a homotrimer or heterotrimer, upon recognition of the extramembrane signal. In some embodiments, the receptor polypeptide is configured to form a tetramer, e.g., a homotetramer or heterotetramer, upon recognition of the extramembrane signal.

The provided chimeric transmembrane receptor polypeptide can be configured to recognize one or more of a wide variety of extramembrane signals inducing receptor oligomerization. In some embodiments, the extramembrane signal includes a ligand capable of binding to the extramembrane domain of the chimeric transmembrane receptor polypeptide. In some embodiments, the extramembrane signal includes a ligand that is a small molecule, e.g., a small molecule drug or a small molecule hormone. In some embodiments, the extramembrane signal consists of a small molecule. As used herein, the term “small molecule” refers to a chemical entity having a molecular weight less than 3,000 Daltons. The chimeric transmembrane receptor polypeptide can be configured to recognize, for example, rimiducid (AP1903), darunavir, tamoxifen, estradiol, AP20187, or a symmetrical steroid. The small molecule ligand recognized by the chimeric transmembrane polypeptide can be, for example, a monomer (e.g., caffeine or AP21967), a dimer (e.g., AP20187 or a synthetic Mcl-1 molecular glue), or a tetramer (e.g., a synthetic biotin dendrimer).

In some embodiments, the extramembrane signal includes a ligand that is a metabolite. In some embodiments, the extramembrane signal consists of a metabolite. As used herein, the term “metabolite” refers to a chemical entity produced by one or more enzymatic or non-enzymatic reactions as a result of exposure of an organism to a chemical substance. The chimeric transmembrane receptor polypeptide can be configured to recognize, for example, a cytokine, a chemokine, or a cancer-associated antigen. The receptor polypeptide can be configured to recognize a tumor marker derived from mucin-1 such as Carcinoma Antigen (CA) 15-3. The receptor polypeptide can be configured to recognize a tumor marker derived from mucin-16 such as CA 125. The receptor polypeptide can be configured to recognize amino acid metabolites such as Kynurenine.

In some embodiments, the extramembrane signal includes a ligand that is an oligonucleotide. In some embodiments, the extramembrane signal consists of an oligonucleotide. As used herein, the term “oligonucleotide” refers to a short nucleic acid molecule comprised of at least six covalently linked natural or chemically modified nucleosides. The chimeric transmembrane receptor polypeptide can be configured to recognize an oligonucleotide comprising any combination of DNA nucleotides, RNA nucleotides, locked nucleic acid (LNA) nucleotides, or other synthetic nucleotide derivatives. In some embodiments, the extramembrane signal includes or consists of a ligand that is a longer polynucleotide. The oligonucleotide of the extramembrane signal can include, for example, DNA origami creating Y-DNA, X-DNA, and/or I-DNA. The oligonucleotide of the extramembrane signal can have a multimeric form, such as in a DNA origami structure. The oligonucleotide can have a hybrid form in which it is conjugated to a small molecule, such as biotin.

In some embodiments, the extramembrane signal includes a ligand that is a peptide or protein. In some embodiments, the extramembrane signal consists of a ligand that is a peptide or protein. As used herein, the terms “peptide” and “protein” refer to polymers comprised of covalently linked natural or chemically modified amino acid residues. The chimeric transmembrane receptor polypeptide can be configured to recognize, for example, cyclic peptides or linear peptides. The peptide of the extramembrane signal can be a naturally occurring protein, such as EGF. The peptide of the extramembrane signal can be a synthetic an non-naturally occurring protein.

In some embodiments, the extramembrane signal includes a ligand that is a polysaccharide. In some embodiments, the extramembrane signal consists of a ligand that is a polysaccharide. As used herein, the term “polysaccharide” refers to a polymer comprised of covalently linked natural or chemically modified sugar molecules. Polysaccharides include, for example, cellulose, hemicellulose, lignocellulose, starch, and the like.

In some embodiments, the extramembrane signal includes a ligand that is a lipid. In some embodiments, the extramembrane signal consists of a ligand that is a lipid. As used herein, the term “lipid” refers to a chemical entity having a hydrophilic moiety covalently attached to one or more hydrophobic moieties. Lipid molecules include, for example, fats, waxes, steroids, cholesterol, fat-soluble vitamins, monoglycerides, diglycerides, phospholipids, sphingolipids, glycolipids, cationic or anionic lipids, derivatized lipids, and the like. The receptor polypeptide can be configured to recognize modified lipids, e.g., lipids modified as a result of a cancer.

In some embodiments, the extramembrane signal includes a ligand that is an antibody. In some embodiments, the extramembrane signal consists of a ligand that is an antibody. As used herein, the term “antibody” refers to a polypeptide of the immunoglobulin family or a polypeptide comprising fragments of an immunoglobulin that is capable of non-covalently, reversibly, and in a specific manner binding to an epitope of a corresponding antigen. The term includes, but is not limited to, polyclonal or monoclonal antibodies of the isotype classes IgA, IgD, IgE, IgG, and IgM, derived from human or other mammalian cells, including natural or genetically modified forms such as humanized, human, single-chain, chimeric, synthetic, recombinant, hybrid, mutated, grafted, and in vitro generated antibodies. The term encompasses conjugates, including but not limited to fusion proteins containing an immunoglobulin moiety, e.g., chimeric or bispecific antibodies, and fragments such as Fab, F(ab′)2, Fv, scFv, Fd, dAb and other compositions. The chimeric transmembrane receptor polypeptide can be configured to recognize, for example, nivolumab, ipilimumab, or tocilizumab.

In some embodiments, the extramembrane signal includes a ligand that is a nanobody. In some embodiments, the extramembrane signal consists of a ligand that is a nanobody. As used herein, the terms “nanobody” or “single-domain antibody” refer to an antibody fragment comprised of a single monomeric variable antibody domain, having a molecular weight of less than 20 kDa, and able to bind selectively to a specific antigen.

In some embodiments, the extramembrane signal includes a ligand that is a single-chain variable fragment (scFv). In some embodiments, the extramembrane signal consists of a ligand that is an scFv. As used herein, the terms “single-chain variable fragment” and “scFv” refer to an antibody in which the variable domains of the heavy chain and of the light chain of a traditional two chain antibody have been joined to form one chain. The terms can also refer to antibodies in which a linker peptide is inserted between the heavy and light chains to allow for proper folding of the single chain and creation of an active binding site.

In some embodiments, the extramembrane signal includes or consists of a change in an environmental parameter, e.g., a change in the extramembrane environment proximate to the provided chimeric transmembrane receptor polypeptide. For example, in some embodiments, the extramembrane signal includes or consists of a change in temperature. e.g., a change in the temperature of the extramembrane environment proximate to the chimeric transmembrane receptor polypeptide. The extramembrane signal can include or consist of an increase in the extramembrane temperature that is at least as large as a predetermined amount. The extramembrane signal can include or consist of a decrease in the extramembrane temperature that is at least as large as a predetermined amount. In some embodiments, the extramembrane signal includes or consists of a change in pH, e.g., a change in the pH of the extramembrane environment proximate to the chimeric transmembrane receptor polypeptide. The extramembrane signal can include or consist of an increase in the extramembrane pH that is at least as large as a predetermined amount. The extramembrane signal can include or consists of a decrease in the extramembrane pH that is at least as large as a predetermined amount.

In some embodiments, the extramembrane signal includes or consists of a change in sound, e.g., a change in the frequency, amplitude, envelope, or other property of extramembrane sound impinging on the chimeric transmembrane receptor polypeptide. The extramembrane signal can include or consist of sound, e.g., acoustic sound, ultrasound, and/or infrasound, directed at the receptor polypeptide. In some embodiments, the extramembrane signal includes or consists of a change in electromagnetic radiation, e.g., a change in the frequency, amplitude, or other property of extramembrane electromagnetic radiation impinging on the chimeric transmembrane receptor polypeptide. The extramembrane signal can include or consist of electromagnetic radiation, e.g., visible light, infrared light, ultraviolet light, X-rays, radio waves, and/or gamma rays, directed at the receptor polypeptide. In some embodiments, the extramembrane signal includes or consists of a change in mechanical force impinging on the chimeric transmembrane receptor polypeptide. The extramembrane signal can include or consist of a mechanical force directed at the receptor polypeptide.

In some embodiments, the extramembrane domain of the provided chimeric transmembrane receptor polypeptide includes an FK506 (FKBP) binding protein family domain, or a variant or fragment thereof. In some embodiments, the extramembrane domain consists of an FKBP binding family domain, or a variant or fragment thereof. The chimeric transmembrane receptor polypeptide extramembrane domain can include or consist of a homodimer FKBP domain. The chimeric transmembrane receptor polypeptide extramembrane domain can include or consist of a heterodimer FKBP/FRB domain. As used herein, the terms “variant,” and “fragment,” refer to a polypeptide related to a wild-type polypeptide, for example, either by amino acid sequence, structure (e.g., secondary and/or tertiary), activity (e.g., enzymatic activity) and/or function. Variants and fragments of a polypeptide can include one or more amino acid variations (e.g., mutations, insertions, and deletions), truncations, modifications, or combinations thereof compared to a wild-type polypeptide. A variant or fragment can include at 50%, e.g., at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% of the sequence, structure, activity, and/or function of the corresponding wild-type polypeptide.

In some embodiments, the extramembrane domain of the provided chimeric transmembrane receptor polypeptide includes a bromodomain and extra terminal domain (BET) family domain, or a variant or fragment thereof. In some embodiments, the extramembrane domain consists of a BET family domain, or a variant or fragment thereof. BET family domains suitable for use with the extramembrane domain of the chimeric transmembrane receptor polypeptide include, for example, bromodomain-containing protein 2 (BRD2), BRD3, BRD4, and bromodomain testis-specific protein (BRDT).

In some embodiments, the extramembrane domain of the provided chimeric transmembrane receptor polypeptide includes a B-cell lymphoma 2 (Bcl-2) family domain, or a variant or fragment thereof. In some embodiments, the extramembrane domain consists of a Bcl-2 family domain, or a variant or fragment thereof. Bcl-2 family domains suitable for use with the extramembrane domain of the chimeric transmembrane receptor polypeptide include, for example, Bcl-XL, Bcl-2-like 1 (BCL2L1), BCL2L2, BCL2L10, BCL2L13, BCL2L14, Bcl-2 related ovarian killer (BOK), induced myeloid leukemia cell differentiation protein Mcl-1, Mcl-2, Bim, Bid, BAD, cell death abnormality gene 9 (CED-9), Bcl-2-related protein A1, Bfl-1, Bcl-2-associated X protein (Bax), Bcl-2 homologous antagonist/killer (Bak), Diva, Bcl-Xs, and Egl-1.

In some embodiments, the extramembrane domain of the provided chimeric transmembrane receptor polypeptide includes a soluble N-ethylmaleimide-sensitive factor (NSF) attachment protein (SNAP) receptor domain, or a variant or fragment thereof. In some embodiments, the extramembrane domain consists of a SNAP receptor domain, or a variant or fragment thereof. SNAP receptor domains suitable for use with the extramembrane domain of the chimeric transmembrane receptor polypeptide include, for example, SNAP-TAG®, CLIP-TAG®, ACP-TAG®, and MCP-TAG®. The extramembrane domain can work similarly with other small molecules plus protein via covalent bond formation.

In some embodiments, the extramembrane domain of the provided chimeric transmembrane receptor polypeptide includes a Toll-like receptor (TLR) family domain, or a variant or fragment thereof. In some embodiments, the extramembrane domain consists of a TLR family domain, or a variant or fragment thereof. TLR family domains suitable for use with the extramembrane domain of the chimeric transmembrane receptor polypeptide include, for example, domains from human TLR1, human TLR2, human TLR3, human TLR4, human TLR5, human TLR6, human TLR7, human TLR8, human TLR9, and human TLR10. The TLR family domain of the chimeric transmembrane receptor polypeptide extramembrane domain can be, for example, a domain from murine TLR1, murine TLR2, murine TLR3, murine TLR4, murine TLR5, murine TLR6, murine TLR7, murine TLR8, murine TLR9, murine TLR11 murine TLR12, or murine TLR13. The TLR family domain of the chimeric transmembrane receptor polypeptide extramembrane domain can be from an invertebrate TLR.

In some embodiments, the extramembrane domain of the provided chimeric transmembrane receptor polypeptide includes a receptor tyrosine kinase (RTK) domain, or a variant or fragment thereof. In some embodiments, the extramembrane domain consists of an RTK domain, or a variant or fragment thereof. RTK domains suitable for use with the extramembrane domain of the chimeric transmembrane receptor polypeptide include, for example, domains from the ErbB receptor family (also referred to as the epidermal growth factor receptor (EGFR) family), the insulin receptor family, the platelet-derived growth factor (PDGF) receptor family, the vascular endothelial growth factor (VEGF) receptor family, the fibroblast growth factor (FGF) receptor family, the colon carcinoma kinase (CCK) receptor family, the NGF receptor family, the hepatocyte growth factor (HGF) receptor family, the Eph receptor family, the AXL receptor family, the TIE receptor family, the RYK receptor family, the discoidin domain receptor (DDR) family, the RET receptor family, the ROS receptor family, the leukocine tryrosine kinase (LTK) receptor family, the receptor tyrosine kinase-like orphan receptor (ROR) family, the MuSK receptor family, the LMR receptor family, and the RTK class XX family.

In some embodiments, the extramembrane domain of the provided chimeric transmembrane receptor polypeptide includes an ErbB receptor family domain, or a variant or fragment thereof. In some embodiments, the extramembrane domain consists of an ErbB receptor family domain, or a variant or fragment thereof. ErbB receptor family domains suitable for use with the extramembrane domain of the chimeric transmembrane receptor polypeptide include, for example, domains from Her1 (also referred to as EGFR or ErbB-1), Her2 (also referred to as proto-oncogene Neu, ErbB-2, or CD340), Her3 (also referred to as ErbB-3), and Her 4 (also referred to as ErbB-4).

In some embodiments, the extramembrane domain of the provided chimeric transmembrane receptor polypeptide includes a type I cytokine receptor family domain, or a variant or fragment thereof. In some embodiments, the extramembrane domain consists of a type I cytokine receptor family domain, or a variant or fragment thereof. Type I cytokine receptor family domains suitable for use with the extramembrane domain of the chimeric transmembrane receptor polypeptide include, for example, domains from interleukin (IL) receptors such as IL-1 receptor, IL-2 receptor, IL-3 receptor, IL-4 receptor, IL-5 receptor, IL-6 receptor, IL-7 receptor, IL-9 receptor, IL-11 receptor, IL-12 receptor, IL-13 receptor, IL-15 receptor, IL-18 receptor, IL-21 receptor, IL-23 receptor, and IL-27 receptor. Suitable type I cytokine receptor family domains include those from, for example, colony stimulating factor (CSF) receptors such as erythropoietin receptor, granulocyte-macrophage CSF receptor, granulocyte CSF receptor, and thrombopoietin receptor. Suitable type I cytokine receptor family domains include those from, for example, a hormone receptor/neuropeptide receptor such as growth hormone receptor, prolactin receptor, and leptin receptor. The type I cytokine receptor family domain can include a domain from common gamma chain (also referred to as CD132), common beta chain (also referred to as CD131), or glycoprotein 130 (also referred to as CD130).

In some embodiments, the extramembrane domain of the provided chimeric transmembrane receptor polypeptide includes a type Il cytokine receptor family domain, or a variant or fragment thereof. In some embodiments, the extramembrane domain consists of a type II cytokine receptor family domain, or a variant or fragment thereof. Type II cytokine receptor family domains suitable for use with the extramembrane domain of the chimeric transmembrane receptor polypeptide include, for example, domains from interferon (IFN) receptors such as IFN-α/β receptor, IFN-γ receptor, and IFN type III receptor. Suitable type II cytokine receptor family domains include those from, for example, IL receptors such as IL-10 receptor, IL-20 receptor, IL-22 receptor, and IL-28 receptor.

In some embodiments, the extramembrane domain of the provided chimeric transmembrane receptor polypeptide includes a transforming growth factor beta (TGFβ) receptor family domain, or a variant or fragment thereof. In some embodiments, the extramembrane domain consists of a TGFβ receptor family domain, or a variant or fragment thereof. TGFβ receptor family domains suitable for use with the extramembrane domain of the chimeric transmembrane receptor polypeptide include, for example, domains from TGFβ type I receptors such as activin receptor-like kinase 1 (ALK1, also referred to as ACVRL1), ALK2 (also referred to as ACVR1A), ALK3 (also referred to as BMPR1A), ALK4 (also referred to as ACVR1B), ALK5 (also referred to as TGFβR1), ALK6 (also referred to as BMPR1B), and ALK7 (also referred to as ACVR1C). Suitable TGFβ receptor family domains include those from, for example, TGFβ type II receptors such as TGFβR2, bone morphogenetic protein receptor type II (BMPR2), activing receptor type-2A (ACVR2A), ACVR2B, and Anti-Mullerian hormone receptor type 2 (AMHR2). Suitable TGFβ receptor family domains include those from, for example, TGFβ type III receptors such as TGFβR3 (also referred to as β-glycan).

In some embodiments, the extramembrane domain of the provided chimeric transmembrane receptor polypeptide includes a tumor necrosis factor (TNF) receptor family domain, or a variant or fragment thereof. In some embodiments, the extramembrane domain consists of a TNF receptor family domain, or a variant or fragment thereof. TNF receptor family domains suitable for use with the extramembrane domain of the chimeric transmembrane receptor polypeptide include, for example, domains from TNF receptor superfamily 1A (TNFRSF1A, also referred to as CD120a), THFRSF1B (also referred to as CD120b), TNFRSF2 (also referred to as tumor necrosis factor or TNFα), TNFRSF3 (also referred to as lymphotoxin beta or TNFγ), TNFRSF4 (also referred to as OX40 ligand, CD252, or CD134L), TNFRSF5 (also referred to as CD40 ligand or CD154), TNFRSF6 (also referred to as Fas ligand, CD178, or CD95L), TNFRSF7 (also referred to as CD27 ligand or CD70), TNFRSF8 (also referred to as CD30 ligand or CD153), TNFRSF9 (also referred to as CD137 ligand or 4-1 BBL), TNFRSF10 (also referred to as TRAIL or CD243), TNFRSF11 (also referred to as RANKL or CD254), TNFRSF12 (also referred to as TWEAK), TNFRSF13 (also referred to as APRIL or CD256), TNFRSF13b (also referred to as BAFF or CD257), TNFRSF14 (also referred to as LIGHT or CD258), TNFRSF15 (also referred to as VEGI), TNFRSF18, and TNFRSF19 (also referred to as ectodysplasin A).

In some embodiments, the extramembrane domain of the provided chimeric transmembrane receptor polypeptide includes an immunoglobulin superfamily (IgSF) domain, or a variant or fragment thereof. In some embodiments, the extramembrane domain consists of an IgSF domain, or a variant or fragment thereof. IgSF domains suitable for use with the extramembrane domain of the chimeric transmembrane receptor polypeptide include, for example, domains from antigen receptors such as IgA, IgD, IgE, IgG, IgM, and T-cell receptor chains. Suitable IgSF domains include those from, for example, antigen presenting molecules such as class I major histocompatibility complex (MHC), class II MHC, and β-2 microglobulin. Suitable IgSF domains include those from, for example, co-receptors such as CD4, CD8, and CD19. Suitable IgSF domains include those from, for example, antigen receptor accessory molecules such as CD3, CD79a, and CD79b. Suitable IgSF domains include those from, for example, co-stimulatory or inhibitory molecules such as CD28, CD80, and CD86. Suitable IgSF domains include, for example, killer-cell immunoglobulin-like receptors (KIR) and leukocyte immunoglobulin-like receptors (LLIR). Suitable IgSF domains include, for example, cell adhesion molecules (CAMs) such as neural cell adhesion molecules (NCAMs), intercellular adhesion molecule-1 (ICAM-1), and CD2. Suitable IgSF domains include, for example, those from growth factor receptors such as platelet-derived growth factor receptor (PDGFR) and mast/stem cell growth factor receptor precursor (SCFR). Suitable IgSF domains include, for example, those from receptor tyrosine kinases/phosphatases such as tyrosine-protein kinase receptor Tie-1 precursor, type IIa receptor protein tyrosine phosphatases (RPTPs), and type IIb RPTPs. Suitable IgSF domains include, for example, those from Ig binding receptors such as polymeric immunoglobulin receptor (PIGR). Suitable IgSF domains include, for example, those from cytoskeleton molecules such as myotilin, myopalladin, paladin, titn, obscurin, myomesin-1, and myomesin-2. Suitable IgSF domains include, for example, CD147, CD90, CD7, and butyrophilins.

In some embodiments, the extramembrane domain of the provided chimeric transmembrane receptor polypeptide includes a tropomyosin receptor kinase (trk) family domain, or a variant or fragment thereof. In some embodiments, the extramembrane domain consists of a trk family domain, or a variant or fragment thereof. Trk family domains suitable for use with the extramembrane domain of the chimeric transmembrane receptor polypeptide include, for example, domains from trkA, trkB, and trkC.

In some embodiments, the extramembrane domain of the provided chimeric transmembrane receptor polypeptide includes a glial cell-derived neurotrophic factor (GDNF) receptor family domain, or a variant or fragment thereof. In some embodiments, the extramembrane domain consists of a GDNF receptor family domain, or a variant or fragment thereof. GDNF receptor family domains suitable for use with the extramembrane domain of the chimeric transmembrane receptor polypeptide include, for example, domains from GDNF family receptor alpha-1 (GFRα1), GFRα2, GFRα3, and GFRα4.

In some embodiments, the extramembrane domain of the provided chimeric transmembrane receptor polypeptide includes or consists of an immune signaling domain such as a domain from 4-1BB, or a variant or fragment thereof. In some embodiments, the extramembrane domain consists of an immune signaling domain, or a variant or fragment thereof. In some embodiments, the extramembrane domain of the provided chimeric transmembrane receptor polypeptide includes or consists of a gibberellin-insensitive dwarf (GID) family domain, or a variant or fragment thereof. In some embodiments, the extramembrane domain of the provided chimeric transmembrane receptor polypeptide includes or consists of a biotin receptor domain, or a variant or fragment thereof. In some embodiments, the extramembrane domain of the provided chimeric transmembrane receptor polypeptide includes or consists of an epidermal growth factor receptor domain, or a variant or fragment thereof. In some embodiments, the extramembrane domain of the provided chimeric transmembrane receptor polypeptide includes or consists of an estrogen receptor domain, or a variant or fragment thereof. In some embodiments, the extramembrane domain of the provided chimeric transmembrane receptor polypeptide includes or consists of an androgen receptor domain, or a variant or fragment thereof. In some embodiments, the extramembrane domain of the provided chimeric transmembrane receptor polypeptide includes or consists of an insulin receptor domain, or a variant or fragment thereof. In some embodiments, the extramembrane domain of the provided chimeric transmembrane receptor polypeptide includes or consists of a programmed cell death protein-1 (PD-1) domain, a programmed death-ligand 1 (PD-L1) domain, or a variant or fragment thereof. In some embodiments, the extramembrane domain of the provided chimeric transmembrane receptor polypeptide includes or consists of an AXL receptor tyrosine kinase domain, or a variant or fragment thereof. In some embodiments, the extramembrane domain of the provided chimeric transmembrane receptor polypeptide includes or consists of a single-chain variable fragment (scFv) domain, or a variant or fragment thereof. In some embodiments, the extramembrane domain of the provided chimeric transmembrane receptor polypeptide includes or consists of a nanobody domain, or a variant or fragment thereof.

In some embodiments, the transmembrane domain of the provided chimeric transmembrane receptor polypeptide includes or consists of a TLR family domain, or a variant or fragment thereof. The TLR family domain of the transmembrane domain can be, for example, any of those previously disclosed herein. In some embodiments, the transmembrane domain of the chimeric transmembrane receptor polypeptide includes or consists of an RTK family domain, e.g., an ErbB receptor family domain, or a variant or fragment thereof. The RTK or ErbB receptor family domain of the transmembrane domain can be, for example, any of those previously disclosed herein. In some embodiments, the transmembrane domain of the chimeric transmembrane receptor polypeptide includes or consists of a type I cytokine receptor family domain, or a variant or fragment thereof. The type I cytokine receptor family domain of the transmembrane domain can be, for example, any of those previously disclosed herein. In some embodiments, the transmembrane domain of the chimeric transmembrane receptor polypeptide includes or consists of a type II cytokine receptor family domain, or a variant or fragment thereof. The type II cytokine receptor family domain of the transmembrane domain can be, for example, any of those previously disclosed herein. In some embodiments, the transmembrane domain of the chimeric transmembrane receptor polypeptide includes or consists of a TGFβ receptor family domain, or a variant or fragment thereof. The TGFβ family domain of the transmembrane domain can be, for example, any of those previously disclosed herein. In some embodiments, the transmembrane domain of the chimeric transmembrane receptor polypeptide includes or consists of a TNF receptor family domain, or a variant or fragment thereof. The TNF receptor family domain of the transmembrane domain can be, for example, any of those previously disclosed herein. In some embodiments, the transmembrane domain of the chimeric transmembrane receptor polypeptide includes or consists of an IgSF domain, or a variant or fragment thereof. The IgSF domain of the transmembrane domain can be, for example, any of those previously disclosed herein. In some embodiments, the transmembrane domain of the chimeric transmembrane receptor polypeptide includes or consists of a trk family domain, or a variant or fragment thereof. The trk family domain of the transmembrane domain can be, for example, any of those previously disclosed herein. In some embodiments, the transmembrane domain of the chimeric transmembrane receptor polypeptide includes or consists of a GDNF receptor family domain, or a variant or fragment thereof. The GDNF receptor family domain of the transmembrane domain can be, for example, any of those previously disclosed herein.

In some embodiments, the intramembrane domain of the provided chimeric transmembrane receptor polypeptide includes or consists of a TLR family domain, or a variant or fragment thereof. The TLR family domain of the intramembrane domain can be, for example, any of those previously disclosed herein. In some embodiments, the intramembrane domain of the chimeric transmembrane receptor polypeptide includes or consists of an RTK family domain, e.g., an ErbB receptor family domain, or a variant or fragment thereof. The RTK or ErbB receptor family domain of the intramembrane domain can be, for example, any of those previously disclosed herein. In some embodiments, the intramembrane domain of the chimeric transmembrane receptor polypeptide includes or consists of a type I cytokine receptor family domain, or a variant or fragment thereof. The type I cytokine receptor family domain of the intramembrane domain can be, for example, any of those previously disclosed herein. In some embodiments, the intramembrane domain of the chimeric transmembrane receptor polypeptide includes or consists of a type II cytokine receptor family domain, or a variant or fragment thereof. The type II cytokine receptor family domain of the intramembrane domain can be, for example, any of those previously disclosed herein. In some embodiments, the intramembrane domain of the chimeric transmembrane receptor polypeptide includes or consists of a TGFβ receptor family domain, or a variant or fragment thereof. The TGFβ family domain of the intramembrane domain can be, for example, any of those previously disclosed herein. In some embodiments, the intramembrane domain of the chimeric transmembrane receptor polypeptide includes or consists of a TNF receptor family domain, or a variant or fragment thereof. The TNF receptor family domain of the intramembrane domain can be, for example, any of those previously disclosed herein. In some embodiments, the intramembrane domain of the chimeric transmembrane receptor polypeptide includes or consists of an IgSF domain, or a variant or fragment thereof. The IgSF domain of the intramembrane domain can be, for example, any of those previously disclosed herein. In some embodiments, the intramembrane domain of the chimeric transmembrane receptor polypeptide includes or consists of a trk family domain, or a variant or fragment thereof. The trk family domain of the intramembrane domain can be, for example, any of those previously disclosed herein. In some embodiments, the intramembrane domain of the chimeric transmembrane receptor polypeptide includes or consists of a GDNF receptor family domain, or a variant or fragment thereof. The GDNF receptor family domain of the intramembrane domain can be, for example, any of those previously disclosed herein.

In some embodiments, the intramembrane domain of the provided chimeric transmembrane receptor polypeptide includes a gene editing nuclease domain, or a variant or fragment thereof. In some embodiments, the intramembrane domain consists or a gene editing domain, or a variant or fragment thereof. Gene editing domains suitable for use with the intramembrane domain of the chimeric transmembrane receptor polypeptide include, for example, clustered regularly interspaced short palindromic repeats (CRISPR), Zinc finger nuclease, transcription activator-like effector nuclease (TALEN), CRISPR associated protein 9 (Cas9), Cas12, Cas13, Cas14, CasX, CasY, Caso, base editor, and primer editor. In some embodiments, the intramembrane domain includes or consist of an epigenetic editor domain, or a variant or fragment thereof.

In some embodiments, the intramembrane domain of the provided chimeric transmembrane receptor polypeptide includes a transcriptional controller domain, or a variant of fragment thereof. In some embodiments, the intramembrane domain consists of a transcriptional controller domain, or a variant or fragment thereof. Transcriptional controller domains suitable for use with the intramembrane domain of the chimeric transmembrane receptor polypeptide include, for example, CRISPRi/a, Zinc finger, TALE, dCas9, dCas12, dCas13, dCas14, dCasX, dCasY, and dCasφ. The transcriptional controller domain can be used itself or fused to many other transcriptional or epigenetic effector domains.

In some embodiments, the intramembrane domain of the provided chimeric transmembrane receptor polypeptide includes an RNA controller domain, or a variant of fragment thereof. In some embodiments, the intramembrane domain consists of an RNA controller domain, or a variant or fragment thereof. RNA controller domains suitable for use with the intramembrane domain of the chimeric transmembrane receptor polypeptide include, for example, Cas13, Cas14, CasΦ, ADARs, Argonaute, and engineered variants thereof.

In some embodiments, the intramembrane domain of the provided chimeric transmembrane receptor polypeptide includes a protein controller domain, or a variant of fragment thereof. In some embodiments, the intramembrane domain consist of a protein controller domain, or a variant or fragment thereof. Protein controller domains suitable for use with the intramembrane domain of the chimeric transmembrane receptor polypeptide include, for example, a protease such as Tobacco Etch Virus (TEV) protease, hepatitis C virus (HCV) protease, and human immunodeficiency virus-1 (HIV-1) protease. Protein controller domains suitable for use with the intramembrane domain include, for example, antiCRISPRs (Acrs) and other post-modification enzymes and degradation complexes.

The intramembrane domain of the provided chimeric transmembrane receptor polypeptide is configured to induce the activation of one or more intramembrane signal pathways upon oligomerization, e.g., dimerization or 3-dimensional (3D) clustering, of the receptor polypeptide. In some embodiments, the intramembrane domain is configured to induce the activation of two intramembrane signal pathways. In some embodiments, the intramembrane domain induces the activation of more than two pathways, e.g., more than three pathways, more than four pathways, more than five pathways, more than six pathways, more than seven pathways, more than eight pathways, more than nine pathways, or more than ten pathways.

In some embodiments, at least one of the one or more intramembrane signal pathways activated by the intramembrane domain is an exogenous pathway. In some embodiments, each of the one or more intramembrane signal pathways activated by the intramembrane domain is an exogenous pathway. In some embodiments, at least one of the one or more intramembrane signal pathways activated by the intramembrane domain is an endogenous pathway. In some embodiments, each of the one or more intramembrane signal pathways activated by the intramembrane domain is an endogenous pathway.

In some embodiments, at least one of the one or more intramembrane signal pathways activated by the intramembrane domain is a synthetic pathway. In some embodiments, each of the one or more intramembrane signal pathways activated by the intramembrane domain is a synthetic pathway. In some embodiments, at least one of the one or more intramembrane signal pathways activated by the intramembrane domain is a naturally occurring pathway. In some embodiments, each of the one or more intramembrane signal pathways activated by the intramembrane domain is a naturally occurring pathway.

A wide variety of intramembrane signal pathways are suitable for activation by a chimeric transmembrane receptor polypeptide as disclosed herein. The one or more intramembrane signal pathways activated by the receptor polypeptide can include, for example, pathways responsible for genome sequence editing, transcription activation or repression, epigenetic modifications, genome translocation and rearrangement, RNA expression or degradation, RNA splicing or processing, post-transcription modifications of mRNA or mRNA, post-translational modifications of proteins, cleavage or proteolysis of proteins, production or degradation of metabolites or other chemistries, trafficking of signaling molecules, cell cycle control, cell differentiation or reprogramming, T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, and/or production of oncolytic virus.

The provided chimeric transmembrane receptor polypeptide can optionally include one or more signal peptides. In some embodiments, the signal peptide of the provided chimeric transmembrane receptor polypeptide is a TLR family signal peptide, e.g., a TLR5 signal peptide, or a variant or fragment thereof. The TLR5 signal peptide can have a sequence of MGDHLDLLLGVVLMAGPVF, or a variant or fragment sequence thereof. In some embodiments, the signal peptide of the chimeric transmembrane receptor polypeptide is a CD3ε signal peptide or a variant or fragment thereof. The CD3ε signal peptide can have a sequence of MQSGTHWRVLGLCLLSVGVWGQD, or a variant or fragment sequence thereof. In some embodiments, the signal peptide of the chimeric transmembrane receptor polypeptide is a CD8α signal peptide or a variant or fragment thereof. The CD8α signal peptide can have a sequence of MALPVTALLLPLALLLHAARP, or a variant or fragment thereof. In some embodiments, the signal peptide of the chimeric transmembrane receptor polypeptide is an IgK signal peptide or a variant or fragment thereof. The IgK signal peptide can have a sequence of METDTLLLWVLLLWVPGSTGD, or a variant or fragment thereof. In some embodiments, the signal peptide of the chimeric transmembrane receptor polypeptide is an IgL signal peptide or a variant or fragment thereof. The IgL signal peptide can have a sequence of MAWTSLILSLLALCSGAS, or a variant or fragment thereof. In some embodiments, the signal peptide of the chimeric transmembrane receptor polypeptide is a mouse CD4 signal peptide or a variant or fragment thereof. The mouse CD4 signal peptide can have a sequence of MCRAISLRRLLLLLLQLSQLLAVTQG, or a variant or fragment thereof.

In some embodiments, the signal peptide of the provided chimeric transmembrane receptor polypeptide includes an oligomerization peptide or a variant or fragment thereof. In some embodiments, the signal peptide includes a pro-clustering peptide or a variant or fragment thereof. In some embodiments, the signal peptide includes an anti-clustering peptide sequence or a variant or fragment thereof. In these ways, the signal peptide can introduce additional forces that influence the oligomerization or clustering of the receptor, and act as positive and/or negative gauges. Exemplary peptides that can influence the receptor oligomerization or clustering include those capable of forming cysteine-cysteine disulfide bonds, charge-charge-based attractions, or charge-charge-based repulsions.

The provided chimeric transmembrane receptor polypeptide can optionally include one or more linker peptide sequences. In some embodiments, the chimeric transmembrane receptor polypeptide includes two linker peptide sequences. In some embodiments, the receptor polypeptide includes more than two linker peptide sequences.

Linker sequences suitable for use with the provided chimeric transmembrane receptor polypeptide include, for example, those consisting of glycine (G) and serine (S). In some embodiments, at least one of the one or more linker peptide sequences of the provided chimeric transmembrane receptor polypeptide is a GGS linker sequence. In some embodiments, each of the one or more linker peptide sequences is GGS. In some embodiments, at least one of the one or more linker peptide sequences of the chimeric transmembrane receptor polypeptide is a GGSGGSGGS linker sequence. In some embodiments, each of the one or more linker peptide sequences is GGSGGSGGS. In some embodiments, at least one of the one or more linker peptide sequences of the chimeric transmembrane receptor polypeptide is a GS linker sequence. In some embodiments, each of the one or more linker peptide sequences is GS. In some embodiments, at least one of the one or more linker peptide sequences of the chimeric transmembrane receptor polypeptide is a GSGSGS linker sequence. In some embodiments, each of the one or more linker peptide sequences is GSGSGS.

Other linker sequences suitable for use with the provided chimeric transmembrane receptor polypeptide include, for example, IgG hinge linker sequences. In some embodiments, at least one of the one or more linker peptide sequences of the chimeric transmembrane receptor polypeptide is a wild-type IgG4 ESKYGPPCPPCP linker sequence. In some embodiments, each of the one or more linker peptide sequences is ESKYGPPCPPCP. In some embodiments, at least one of the one or more linker peptide sequences of the chimeric transmembrane receptor polypeptide is a mutated IgG4 ESKYGPPAPPAP linker sequence. In some embodiments, each of the one or more linker peptide sequences is ESKYGPPAPPAP.

In some embodiments, at least one of the one or more linker peptide sequences of the provided chimeric transmembrane receptor polypeptide includes an oligomerization peptide sequence or a variant or fragment thereof. In some embodiments, at least one of the one or more linker peptide sequences includes a pro-clustering peptide sequence or a variant or fragment thereof. In some embodiments, at least one of the one or more linker peptide sequences includes an anti-clustering peptide sequence or a variant or fragment thereof. In these ways, the linker peptides can introduce additional forces that influence the oligomerization or clustering of the receptor, and act as positive and/or negative gauges. Exemplary sequences that can influence the receptor oligomerization or clustering include those capable of forming cysteine-cysteine disulfide bonds, charge-charge-based attractions, or charge-charge-based repulsions.

In another aspect, this provided chimeric transmembrane receptor polypeptide is a

part of a system further comprising a membrane. The membrane of the system separates two spatial regions, which can be, for example, an extracellular and intracellular region, a cytoplasmic and nuclear region, or regions outside and inside of an intracellular vesicle or organelle. The receptor polypeptide is situated such that the extramembrane domain and the intramembrane domain are located on opposite sides of the membrane, while the intramembrane domain is located within the membrane. In some embodiments, the membrane of the provided system is a cellular membrane. In some embodiments, the membrane of the provided system is a nuclear membrane. In some embodiments. the membrane of the provided system is an organelle membrane. In some embodiments, the membrane of the provided system is a vesicle membrane.

The provided system can further include a trans-acting receptor polypeptide. In some embodiments, the trans-acting receptor polypeptide of the provided system is a chimeric antigen receptor (CAR). In some embodiments, the trans-acting receptor polypeptide of the provided system is a T cell antigen receptor (TCR). In some embodiments, the trans-acting receptor polypeptide of the provided system is a Synthetic Notch (SynNotch) receptor. In some embodiments, the trans-acting receptor polypeptide of the provided system is a GPCR TANGO™ receptor. In some embodiments, the trans-acting receptor polypeptide of the provided system is a CRISPR ChaCha receptor. In some embodiments, the trans-acting receptor polypeptide of the provided system is a B cell receptor (BCR). In some embodiments, the trans-acting receptor polypeptide of the provided system is a C-type lectin-like receptor, e.g., Ly49. In some embodiments, the trans-acting receptor polypeptide of the provided system is a natural cytotoxicity receptor (NCR). In some embodiments, the trans-acting receptor polypeptide of the provided system is an Fc receptor, e.g., CD64.

In another aspect, the provided chimeric transmembrane receptor polypeptide or system is within a host cell. In some embodiments, the host cell expresses the chimeric transmembrane receptor polypeptide. As used herein, the term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, com, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, and the like), seaweeds (e.g., kelp), a fungal cell (e.g., a yeast cell, a cell from a mushroom), a cell from an invertebrate animal (e.g., fruit fly, cnidarian, echinoderm, nematode, mollusk, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Sometimes a cell does not originate from a natural organism (e.g., a cell can be a synthetically made, sometimes termed an artificial cell).

A wide variety of cell types are suitable for use as the provided host cell. In some embodiments, the host cell is an immune cell, including any cell that is involved in an immune response. For example, the use of engineered innate immune cells such as optionally allogenic natural NK cells, iPSC-derived NK cells, and/or macrophage cells can greatly facilitate treatment of solid tumor while avoiding side effects. This allows ligand-mediated precise activation of immunity at a precise location (e.g., presence of a local tumor signal) and at a specific time point (e.g., administration of the drug). This effect is inducible and reversible.

In some embodiments, the host immune cell type includes granulocytes such as asophils, cosinophils, and neutrophils; mast cells; monocytes which can develop into macrophages; antigen-presenting cells such as dendritic cells; and lymphocytes such as natural killer cells (NK cells), B cells, and T cells. In some embodiments, the host immune cell is an immune effector cell. An immune effector cell is an immune cell that can perform a specific function in response to a stimulus. In some embodiments, the host immune cell is an immune effector cell which can induce cell death. In some embodiments, the host immune cell is a lymphocyte. In some embodiments, the lymphocyte is a NK cell. In some embodiments the lymphocyte is a T cell. In some embodiments, the T cell is an activated T cell. T cells include both naive and memory cells (e.g., central memory or T_{CM}, effector memory or T_{EM} and effector memory RA or T_{EMRA}), effector cells (e.g., cytotoxic T cells or CTLs or Tc cells), helper cells (e.g., Th1, Th2, Th3, Th9, Th7, TFH), regulatory cells (e.g., Treg, and Trl cells), natural killer T cells (NKT cells), tumor infiltrating lymphocytes (TILs), lymphocyte-activated killer cells (LAKs), αβ T cells, γδ T cells, and similar unique classes of the T cell lineage.

T cells can be divided into two broad categories: CD8+ T cells and CD4+ T cells, based on which protein is present on the cell's surface. T cells expressing a provided chimeric transmembrane receptor polypeptide can carry out multiple functions, including killing infected cells and activating or recruiting other immune cells. CD8+ T cells are referred to as cytotoxic T cells or cytotoxic T lymphocytes (CTLs). CTLs expressing a provided chimeric transmembrane receptor polypeptide can be involved in recognizing and removing virus-infected cells and cancer cells. CTLs have specialized compartments, or granules, containing cytotoxins that cause apoptosis, e.g., programmed cell death. CD4 T cells can be subdivided into four sub-sets—Th1, Th2, Th17, and Treg, with “Th” referring to “T helper cell,” although additional sub-sets may exist. Th1 cells can coordinate immune responses against intracellular microbes, especially bacteria. They can produce and secrete molecules that alert and activate other immune cells, like bacteria-ingesting macrophages. Th2 cells are involved in coordinating immune responses against extracellular pathogens, like helminths (parasitic worms), by alerting B cells, granulocytes, and mast cells. Th17 cells can produce interleukin 17 (IL-17), a signaling molecule that activates immune and non-immune cells. Th 17 cells are important for recruiting neutrophils.

The receptor can be engineered into regenerative host cell types to direct the ligand-mediated cell differentiation and programming. This allows site-specific differentiation of cells and tissues to repair or regenerate a damaged or aged body. In some embodiments, the host cell is a stem cell. The host cell can be, for example, an induced pluripotent stem cell (iPSC), an embryonic stem cell (ESC), an adult stem cell, or a mesenchymal stem cell (MSC). In some embodiments, the host cell is a progenitor cell. The host cell can be, for example, a neural progenitor cell, a skeletal progenitor cell, a muscle progenitor cell, a fat progenitor cell, a heart progenitor cell, a chondrocyte, or a pancreatic progenitor cell.

In another aspect, a population of host cells is provided. Each host cell of the population independently includes a chimeric transmembrane receptor as disclosed herein, or a system as disclosed herein.

### Methods

In another aspect, a method for activating an intramembrane signal pathway is disclosed. The activated intramembrane signal pathway can be any of those disclosed herein. The intramembrane signal pathway activation method includes providing any of the chimeric transmembrane receptor polypeptides as disclosed herein, any of the systems as disclosed herein, any of the host cells as disclosed herein, or any of the populations of host cells as disclosed herein. In some embodiments, the method further includes exposing the chimeric transmembrane receptor to the extramembrane signal. Exposing can be performed for any suitable length of time, for example at least 1 minute, at least 5 minutes, at least 10 minutes, at least 30 minutes, at least 1 hour, at least 2 hours, at least 3 hours, at least 4 hours, at least 5 hours, at least 6 hours, at least 7 hours, at least 8 hours, at least 12 hours, at least 16 hours, at least 20 hours, at least 24 hours, at least 2 days, at least 3 days, at least 4 days, at least 5 days, at least 6 days, at least 1 week, at least 2 weeks, at least 3 weeks, at least 1 month or longer.

In another aspect, a method for preventing or treating a disease in a subject is disclosed. The new class of synthetic receptors can be used according to the provided method for next generation cell therapies. By inducing the receptor by either endogenous (e.g., cytokines, tumor microenvironment signals, antigens) or exogenous (e.g., small molecule drugs, peptides, biologics, ultrasound) inputs and “wiring” the receptor to various cellular pathways, these engineered cell therapy methods can have indications for any malady for which cells are involved in such as cancer, infectious diseases, wound healing, autoimmunity, regenerative medicine, CNS diseases, and anti-aging.

As used herein, the term “treatment” refers to an approach for obtaining beneficial or desired results including but not limited to a therapeutic benefit and/or a prophylactic benefit. A therapeutic benefit imparts any relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested. A treatment can involve any of ameliorating one or more symptoms of disease, e.g., cancer, preventing the manifestation of such symptoms before they occur; slowing down or completely preventing the progression of the disease (as may be evident by longer periods between reoccurrence episodes, slowing down or prevention of the deterioration of symptoms, etc.), enhancing the onset of a remission period, slowing down the irreversible damage caused in the progressive-chronic stage of the disease (both in the primary and secondary stages), delaying the onset of said progressive stage, or any combination thereof.

The provided disease prevention or treatment methods include administering to the subject a therapeutically effective amount of the chimeric transmembrane receptor polypeptides as disclosed herein, the systems as disclosed herein, the host cells as disclosed herein, or the populations of host cells as disclosed herein. As used herein, the term “administering” refers to delivery of agents or compositions to the desired site of biological action. Administration methods include, but are not limited to parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. Other modes of delivery include, but are not limited to, the use of liposomal formulations, intravenous infusion, transplantation, etc. One skilled in the art will know of additional methods for administering a therapeutically effective amount of a composition of the present disclosure for preventing or relieving one or more symptoms associated with a disease

As used herein, the term “therapeutically effective amount” refers to the quantity of a composition that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

Pharmaceutical compositions containing chimeric transmembrane receptor polypeptides, systems, or host cells described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Amounts effective for this use can vary based on the severity and course of the disease or condition, previous therapy, the subject's health status, weight, and response to the drugs, and the judgment of the treating physician.

Chimeric transmembrane receptor polypeptides, systems, or host cells described herein can be administered before, during, or after the occurrence of a disease or condition, and the timing of administering the composition can vary. For example, a pharmaceutical compositions can be used as a prophylactic and can be administered continuously to subjects with a propensity to conditions or diseases in order to prevent the occurrence of the disease or condition. The pharmaceutical compositions can be administered to a subject during or as soon as possible after the onset of the symptoms. The administration can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A composition can be administered as soon as is practicable after the onset of a disease or condition is detected or suspected, and for a length of time necessary for the treatment of the disease, such as, for example, from about 1 month to about 3 months. The length of treatment can vary for each subject.

A wide variety of diseases can be prevented or treated using the provided methods. The invention is suitable for broad cell therapy applications to treat, for example, blood or solid cancer, viral infections, bacterial infections, genetic diseases, wound healing, autoimmunity, regenerative medicine, CNS diseases, and anti-aging.

In some embodiments, the prevented or treated disease is a cancer. Non-limiting examples of cancers that can be treated with the provided chimeric transmembrane receptor polypeptides, systems, or host cells include Acanthoma, Acinic cell carcinoma, Acoustic neuroma, Acral lentiginous melanoma, Acrospiroma, Acute cosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pincoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

In some embodiments, the prevented or treated disease is a cancerous tumor. The cancerous tumor can be a solid cancerous tumor or a liquid cancerous tumor. The liquid cancerous tumor can be, for example, a lymphoma or a leukemia. A tumor treated with the methods disclosed herein can result in stabilized tumor growth (e.g., one or more tumors do not increase more than 1%, 5%, 10%, 15%, or 20% in size, and/or do not metastasize) In some embodiments, a tumor is stabilized for at least about 1, 2, 3, 4, 5, 6, 7, 8, 9, 10, 11, 12, or more weeks. In some embodiments. a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor or the number of tumor cells is reduced by at least about 5%, 10%, 15%, 20%, 25%, 30%, 35%, 40%, 45%, 50%, 55%, 60%, 65%, 70%, 75%, 80%, 85%, 90%, 95% or more. In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

In some embodiments, the prevented or treated disease is an infectious disease. The infectious disease can be, for example, a viral infectious disease. The infectious disease can be, for example, a bacterial infectious disease. In the case of bacterial infections, the innate immune system must recognize specific markers of the microbes in order to clear the pathogen. These pathogen associated molecular patterns are recognized by various receptors, most notably the TLRs that are common to all immune cells and work to activate immune pathways that turn on their bactericidal capacities. In the case of macrophages, recognition of microbial pathogens through a TLR activates their unique ability to engulf the bacteria within themselves and destroy the pathogen by acidification. However, bacteria have mechanisms to evade macrophages by hiding the molecules that cause this activation. For example, during biomedical device implantations such as catheters and pacemakers, bacteria form dense biofilms around themselves to avoid recognition, causing massive infection issues which have inhibited lifesaving technology from moving to the clinic. The provided chimeric transmembrane receptor polypeptide, system, or host cell can “rewire” these TLRs to recognize the constituents of the biofilm itself as opposed to the bacteria, such that macrophages are activated by the evasion mechanisms, destroying the infection and allowing these devices to be more safely implanted.

In some embodiments, the method further includes exposing the administered chimeric transmembrane receptor to the extramembrane signal. The exposing of the chimeric transmembrane receptor to the extramembrane signal can include, for example, introducing a therapeutically effective amount of the extramembrane signal to the subject.

### Embodiments

The following embodiments are contemplated. All combinations of features and embodiment are contemplated.

### Embodiment 1

A chimeric transmembrane receptor polypeptide configured to oligomerize upon recognition of an extramembrane signal by the chimeric transmembrane receptor polypeptide, the chimeric transmembrane receptor polypeptide comprising: an extramembrane domain; a transmembrane domain; and an intramembrane domain, wherein the intramembrane domain is configured to induce activation of one or more intramembrane signal pathways upon oligomerization of the chimeric transmembrane receptor polypeptide.

### Embodiment 2

An embodiment of embodiment 1, wherein the chimeric transmembrane receptor polypeptide is configured to dimerize upon recognition of the extramembrane signal, and wherein the intramembrane domain is configured to induce activation of the one or more intramembrane signal pathways upon dimerization of the chimeric transmembrane receptor polypeptide.

### Embodiment 3

An embodiment of embodiment 1, wherein the extramembrane domain comprises an FK506 binding protein (FKBP) family domain, a bromodomain and extra terminal domain (BET) family domain, a gibberellin-insensitive dwarf (GID) family domain, a B-cell lymphoma 2 (Bcl-2) family domain, or a variant or fragment thereof.

### Embodiment 4

An embodiment of embodiment 1, wherein the extramembrane domain comprises a soluble N-ethylmaleimide-sensitive factor (NSF) attachment protein (SNAP) receptor domain, a biotin receptor domain, an epidermal growth factor receptor domain, an estrogen receptor domain, an androgen receptor domain, an insulin receptor domain, a programmed cell death protein-1 (PD-1) domain, an AXL receptor tyrosine kinase domain, a single-chain variable fragment (scFv) domain, a nanobody domain, or a variant or fragment thereof.

### Embodiment 5

An embodiment of embodiment 1, wherein the extramembrane domain comprises a toll-like receptor (TLR) family domain, an ErbB receptor family domain, a type I cytokine receptor family domain, a type II cytokine receptor family domain, a transforming growth factor beta (TGFβ) receptor family domain, a tumor necrosis factor (TNF) receptor family domain, an immunoglobulin superfamily (IgSF) domain, a tropomyosin receptor kinase (trk) family domain, a glial cell-derived neurotrophic factor (GDNF) receptor family domain, or a variant or fragment thereof.

### Embodiment 6

An embodiment of any of the embodiments of embodiment 1-5, wherein the transmembrane domain comprises a TLR family domain, an ErbB receptor family domain, a type I cytokine receptor family domain, a type II cytokine receptor family domain, a TGFβ receptor family domain, a TNF receptor family domain, an IgSF domain, a trk family domain, a GDNF receptor family domain, or a variant or fragment thereof.

### Embodiment 7

An embodiment of any of the embodiments of embodiment 1-6, wherein the intramembrane domain comprises a TLR family domain, an ErbB receptor family domain, a type I cytokine receptor family domain, a type Il cytokine receptor family domain, a TGFβ receptor family domain, a TNF receptor family domain, an IgSF domain, a trk family domain, a GDNF receptor family domain, a gene editing nuclease domain, a transcriptional controller domain, an RNA controller domain, a protein controller domain, or a variant or fragment thereof.

### Embodiment 8

An embodiment of any of the embodiments of embodiment 1-7, further comprising: one or more additional extramembrane domains.

### Embodiment 9

An embodiment of any of the embodiments of embodiment 1-8, further comprising: one or more additional intramembrane domains.

### Embodiment 10

An embodiment of any of the embodiments of embodiment 1-9, further comprising: a signal peptide.

### Embodiment 11

An embodiment of embodiment 10, wherein the signal peptide comprises a TLR family signal peptide, a CD3ε signal peptide, a CD8α signal peptide, an IgK signal peptide, an IgL signal peptide, a mouse CD4 signal peptide, or a variant or fragment thereof.

### Embodiment 12

An embodiment of embodiment 10, wherein the signal peptide comprises an oligomerization peptide, a pro-clustering peptide, an anti-clustering peptide, or a variant or fragment thereof.

### Embodiment 13

An embodiment of any of the embodiments of embodiment 1-12, further comprising: one or more linker peptide sequences

### Embodiment 14

An embodiment of embodiment 13, wherein the one or more linker peptide sequences comprise a GGS linker sequence, a GGSGGSGGS linker sequence, a GS linker sequence, a GSGSGS linker sequence, an ESKYGPPAPPAP (mutant IgG4 hinge) linker sequence, an ESKYGPPCPPCP (IgG4 hinge), or a combination thereof.

### Embodiment 15

An embodiment of embodiment 13, wherein the one or more linker peptide sequences include an oligomerization peptide sequence, a pro-clustering peptide sequence, an anti-clustering peptide sequence, or a variant or fragment thereof.

### Embodiment 16

An embodiment of any of the embodiments of embodiment 1-15, wherein the extramembrane signal comprises a ligand capable of binding to the extramembrane domain.

### Embodiment 17

An embodiment of embodiment 16, wherein the ligand comprises a small molecule.

### Embodiment 18

An embodiment of embodiment 16, wherein the ligand comprises an oligonucleotide.

### Embodiment 19

An embodiment of embodiment 16, wherein the ligand comprises a peptide, a protein, a polysaccharide, a lipid, or a combination thereof.

### Embodiment 20

An embodiment of embodiment 16, wherein the ligand comprises an antibody, a nanobody, or an scFv.

### Embodiment 21

An embodiment of embodiment 16, wherein the ligand comprises a metabolite.

### Embodiment 22

An embodiment of any of the embodiments of embodiment 1-15, wherein the extramembrane signal comprises a change in temperature or pH.

### Embodiment 23

An embodiment of any of the embodiments of embodiment 1-15, wherein the extramembrane signal comprises a change in sound or electromagnetic radiation.

### Embodiment 24

An embodiment of any of the embodiments of embodiment 1-15, wherein the extramembrane signal comprises a change in mechanical force.

### Embodiment 25

An embodiment of any of the embodiments of embodiment 1-24, wherein at least one of the one or more intramembrane signal pathways is an exogenous pathway.

### Embodiment 26

An embodiment of any of the embodiments of embodiment 1-24, wherein at least one of the one or more intramembrane signal pathways is a synthetic pathway.

### Embodiment 27

An embodiment of any of the embodiments of embodiment 1-26, wherein the one or more intramembrane signal pathways comprise genome sequence editing, transcription activation or repression, epigenetic modifications, genome translocation and rearrangement, RNA expression or degradation, RNA splicing or processing, post-transcription modifications of mRNA or mRNA, post-translational modifications of proteins, cleavage or proteolysis of proteins, production or degradation of metabolites or other chemistries, trafficking of signaling molecules, cell cycle control, cell differentiation or reprogramming. T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, production of oncolytic virus, or a combination thereof.

### Embodiment 28

A system comprising: a membrane separating an extramembrane region from an intramembrane region: the chimeric transmembrane receptor polypeptide of any of the embodiments of embodiment 1-27, wherein the extramembrane domain is located within the extramembrane region, and wherein the intramembrane domain is located within the intramembrane region.

### Embodiment 29

An embodiment of embodiment 28, wherein the membrane is a cellular membrane, a nuclear membrane, an organelle membrane, or a vesicle membrane.

### Embodiment 30

An embodiment of embodiment 28 or 29, further comprising: a trans-acting receptor polypeptide.

### Embodiment 31

An embodiment of embodiment 30, wherein the trans-acting receptor polypeptide is a chimeric antigen receptor (CAR), a T cell antigen receptor (TCR), a Synthetic Notch (SynNotch) receptor, a GPCR TANGO receptor, a CRISPR ChaCha receptor, a B cell receptor (BCR), C-type lectin-like receptor Ly49, a CD94-NKG2C/E/H heterodimeric receptor, an NKG2D receptor, a DNAM-1/CD226 nectin/nectin-like binding receptor, a CRTAM nectin/netin-like binding receptor, a member of the natural cytotoxicity receptor (NCR) family, a CD64 Fc receptor, a CD32 Fc receptor, a CD16a Fc receptor, a CD16b Fc receptor, a CD23 Fc receptor, a CD89 Fc receptor, a CD351 Fc receptor, an FcεRI Fc receptor, or an FcRn Fc receptor.

### Embodiment 32

An embodiment of any of the embodiments of embodiment A host cell comprising the chimeric transmembrane receptor polypeptide of any of the embodiments of embodiment 1-27 or the system of any of the embodiments of embodiment 28-31.

### Embodiment 33

An embodiment of embodiment 32, wherein the host cell expresses the chimeric transmembrane receptor polypeptide.

### Embodiment 34

An embodiment of embodiment 32 or 33, wherein the host cell is a lymphocyte, a phagocytic cell, a granulocytic cell, or a dendritic cell.

### Embodiment 35

An embodiment of embodiment 34, wherein the lymphocyte is a T cell, a B cell, a natural killer (NK) cell, or an innate lymphoid cell (ILC).

### Embodiment 36

An embodiment of embodiment 35, wherein the T cell is a CD4+ helper αβT cell, a CD8+ killer αβT cell, a δγT cell, or a natural killer T (NKT) cell.

### Embodiment 37

An embodiment of embodiment 34, wherein the phagocytic cell is a monocyte or a macrophage.

### Embodiment 38

An embodiment of embodiment 34, wherein the granulocytic cell is a neutrophil, a basophil, an eosinophil, or a mast cell.

### Embodiment 39

An embodiment of embodiment 32 or 33, wherein the host cell is a stem cell or a progenitor cell.

### Embodiment 40

An embodiment of embodiment 39, wherein the host cell is an induced pluripotent stem cell (iPSC), an embryonic stem cell (ESC), an adult stem cell, or a mesenchymal stem cell (MSC).

### Embodiment 41

An embodiment of embodiment 39, wherein the progenitor cell is a neural progenitor cell, a skeletal progenitor cell, a muscle progenitor cell, a fat progenitor cell, a heart progenitor cell, a chondrocyte, or a pancreatic progenitor cell.

### Embodiment 42

A population of host cells, wherein each host cell of the population independently comprises the chimeric transmembrane receptor polypeptide of any of the embodiments of embodiment 1-27 or the system of any of the embodiments of embodiment 28-31.

### Embodiment 43

A method for activating an intramembrane signal pathway, the method comprising providing the chimeric transmembrane receptor polypeptide of any of the embodiments of embodiment 1-27, the system of any of the embodiments of embodiment 28-31, the host cell of any of the embodiments of embodiment 32-41, or the population of host cells of embodiment 42.

### Embodiment 44

An embodiment of embodiment 43, further comprising exposing the chimeric transmembrane receptor to the extramembrane signal.

### Embodiment 45

A method for preventing or treating a disease in a subject, the method comprising administering to the subject an amount of the chimeric transmembrane receptor polypeptide of any of the embodiments of embodiment 1-27, the system of any of the embodiments of embodiment 28-31, the host cell of any of the embodiments of embodiment 32-41, or the population of host cells of embodiment 42, wherein the amount is therapeutically effective to prevent or treat the disease.

### Embodiment 46

An embodiment of embodiment 45, further comprising, subsequent to the administering, exposing the chimeric transmembrane receptor to the extramembrane signal.

### Embodiment 47

An embodiment of embodiment 46, wherein the exposing comprises introducing to the subject a therapeutically effective amount of the extramembrane signal.

### Embodiment 48

An embodiment of any of the embodiments of embodiment 45-47, wherein the disease is a cancer.

### Embodiment 49

An embodiment of any of the embodiments of embodiment 45-47, wherein the disease is a cancerous tumor.

### Embodiment 50

An embodiment of embodiment 49, wherein the cancerous tumor is a solid cancerous tumor.

### Embodiment 51

An embodiment of embodiment 49, wherein the cancerous tumor is a liquid cancerous tumor.

### Embodiment 52

An embodiment of any of the embodiments of embodiment 45-47, wherein the disease is an infectious disease.

### Embodiment 53

An embodiment of embodiment 52, wherein the infectious disease is a viral infectious disease or a bacterial infectious disease.

### Embodiment 54

An embodiment of any of the embodiments of embodiment 45-47, wherein the disease is an autoimmune disease.

### Embodiment 55

An embodiment of any of the embodiments of embodiment 45-47, wherein the disease is an age-related disease.

### Embodiment 56

A method for healing a wound in a subject, the method comprising administering to the subject an amount of the chimeric transmembrane receptor polypeptide of any of the embodiments of embodiment 1-27, the system of any of the embodiments of embodiment 28-31, the host cell of any of the embodiments of embodiment 32-41, or the population of host cells of embodiment 42, wherein the amount is therapeutically effective to heal the wound.

### Embodiment 57

An embodiment of embodiment 56, further comprising, subsequent to the administering, exposing the chimeric transmembrane receptor to the extramembrane signal.

### Embodiment 58

An embodiment of embodiment 57, wherein the exposing comprises introducing to the subject a therapeutically effective amount of the extramembrane signal.

### EXAMPLES

The present disclosure will be better understood in view of the following non-limiting examples. The following examples are intended for illustrative purposes only and do not limit in any way the scope of the present invention.

In each of the following examples, standard molecular cloning techniques were performed to assemble nucleic acid sequences encoding components of chimeric transmembrane receptor polypeptides onto plasmid backbones. Plasmids were transfected into HEK293T cells to create lentivirus. Lentivirus was transduced into all cell lines to create receptor engineered cells. Presence of the receptor was determined by a fluorescent reporter. Receptor engineered Jurkat cells or Jurkat-NFκβ-GFP and/or Jurkat-NFAT-GFP reporter cells were treated with activator for 8-24 hr. Receptor activity was measured by antibody staining or GFP expression via flow cytometry.

### Example 1

### Extracellular Engineering of Receptor Polypeptides Recognizing Small Molecule Activators

Chimeric transmembrane receptor polypeptides were constructed using a class of natural receptors called the toll-like receptors (TLRs) that possess excellent scaffolding properties. TLRs are naturally membrane-bound, and are found in all arms of life, including bacteria, indicating the ability to be engineered. Furthermore, TLRs exist as monomers that activate by soluble factor induced oligomerization. TLRs are type I transmembrane receptors containing an extracellular domain (ECD) that controls signal binding, a transmembrane domain (TMD) that determines localization, and the intracellular domain (ICD, toll/interleukin-1 receptor (TIR) homology domain) that controls the signal pathway. Table 1 presents information related to different members of the TLR family of receptors.

TABLE 1
TLR family receptors

| | | Ligand | | | |
|---|---|---|---|---|---|
| Receptor | Ligand(s) | location | Adapter(s) | Location | Cell types |
| TLR 1 | multiple triacyl lipopeptides | Bacterial | MyD88/MAL | cell | monocytes/macrophages |
| | | lipoprotein | | surface | a subset of dendritic cells |
| | | | | | B lymphocytes |
| TLR 2 | multiple glycolipids | Bacterial | MyD88/MAL | cell | monocytes/macrophages |
| | | peptidoglycans | | surface | neutrophils |
| | multiple lipopeptides | Bacterial | | | Myeloid dendritic cells |
| | and proteolipids | peptidoglycans | | | Mast cells |
| | lipoteichoic acid | Gram-positive | | | |
| | | bacteria | | | |
| | HSP70 | Host cells | | | |
| | zymosan (Beta-glucan) | Fungi | | | |
| | Numerous others | | | | |
| TLR 3 | double-stranded RNA, poly I:C | viruses | TRIF | cell | Dendritic cells |
| | | | | compartment | B lymphocytes |
| TLR 4 | lipopolysaccharide | Gram-negative | MyD88/MAL/ | cell | monocytes/macrophages |
| | | bacteria | TRIF/TRAM | surface | neutrophils |
| | several heat shock proteins | Bacteria and | | | Myeloid dendritic cells |
| | | host cells | | | Mast cells |
| | fibrinogen | host cells | | | B lymphocytes |
| | heparan sulfate fragments | host cells | | | Intestinal epithelium |
| | hyaluronic acid fragments | host cells | | | Breast cancer cells |
| | nickel | | | | |
| | Various opioid drugs | | | | |
| TLR 5 | Bacterial flagellin | Bacteria | MyD88 | cell | monocyte/macrophages |
| | Profilin | Toxoplasma | | surface | a subset of dendritic cells |
| | | gondii | | | Intestinal epithelium |
| | | | | | Breast cancer cells |
| TLR 6 | multiple diacyl lipopeptides | Mycoplasma | MyD88/MAL | cell | monocytes/macrophages |
| | | | | surface | Mast cells |
| | | | | | B lymphocytes |
| TLR 7 | imidazoquinoline | small synthetic | MyD88 | cell | monocytes/macrophages |
| | loxoribine (a guanosine analogue) | compounds | | compartment | Plasmacytoid dendritic |
| | bropirimine | | | | cells |
| | resiquimod | | | | B lymphocytes |
| | single-stranded RNA | RNA viruses | | | |
| TLR 8 | small synthetic compounds; | | MyD88 | cell | monocytes/macrophages |
| | single-stranded Viral RNA, | | | compartment | a subset of dendritic cells |
| | phagocytized bacterial RNA(24) | | | | Mast cells |
| | | | | | Intestinal epithelial cells |
| | | | | | (IECs) *only |
| | | | | | in Crohn's or ulcerative |
| | | | | | colitis |
| TLR 9 | unmethylated CpG | Bacteria, DNA | MyD88 | cell | monocytes/macrophages |
| | OligodeoxynucleotideDNA | viruses | | compartment | Plasmacytoid dendritic |
| | | | | | cells |
| | | | | | B lymphocytes |
| TLR 10 | triacylated lipopeptides | | unknown | cell | B cells |
| | | | | surface | Intestinal epitelial cells |
| | | | | | monocytes/macrophages |
| TLR 11 | Profilin | Toxoplasma | MyD88 | cell | monocytes/macrophages |
| | | gondii | | compartment^{[34]} | liver cells |
| | | | | | kidney |
| | | | | | urinary |
| | | | | | bladder epithelium |
| TLR 12 | Profilin | Toxoplasma | MyD88 | cell | Neurons |
| | | gondii | | compartment | plasmacytoid dendritic |
| | | | | | cells |
| | | | | | conventional dendritic |
| | | | | | cells |
| | | | | | macrophages |
| TLR 13 | bacterial ribosomal RNA | Virus, bacteria | MyD88, | cell | monocytes/macrophages |
| | sequence “CGGAAAGACC” (but | | TAK-1 | compartment | conventional dendritic |
| | not the methylated version) | | | | cells |

An initial model system focused on TLR5, a cell membrane bound receptor that binds flagellin (a component of bacterial flagellum) and turns on proinflammatory signals. A mutated form of a domain from the homodimerizing human intracellular FK506 rapalog binding domain, FKBP, was connected to the human Toll Like Receptor 5 (hTLR5) transmembrane domain by an IgG4 hinge linker sequence. The FKBP domain can homodimerize (A/A system) with the addition of rapalog AP20187 (bivalent, small molecule). The hTLR5 transmembrane domain was directly connected to the hTLR5 Toll/Il-1 Receptor (TIR) domain. The receptor also had an N-terminal fusion of the signaling peptide from mouse CD4 to improve cellular localization. In this instance, the engineered receptor resides on the cell membrane, where the binding of extracellular rapalog induces homodimerization of the receptor, causing homodimerization of the intracellular TIR domains to activate the MyD88-dependent pathway, a central activation pathway of the innate immune system. The graph of FIG. 2 plots the Nκβ activity for receptor engineered Jurkat-NFκβ-GFP reporter cells treated with AP20187 activator for 8-24 hr, demonstrating activation of the intramembrane signal pathway.

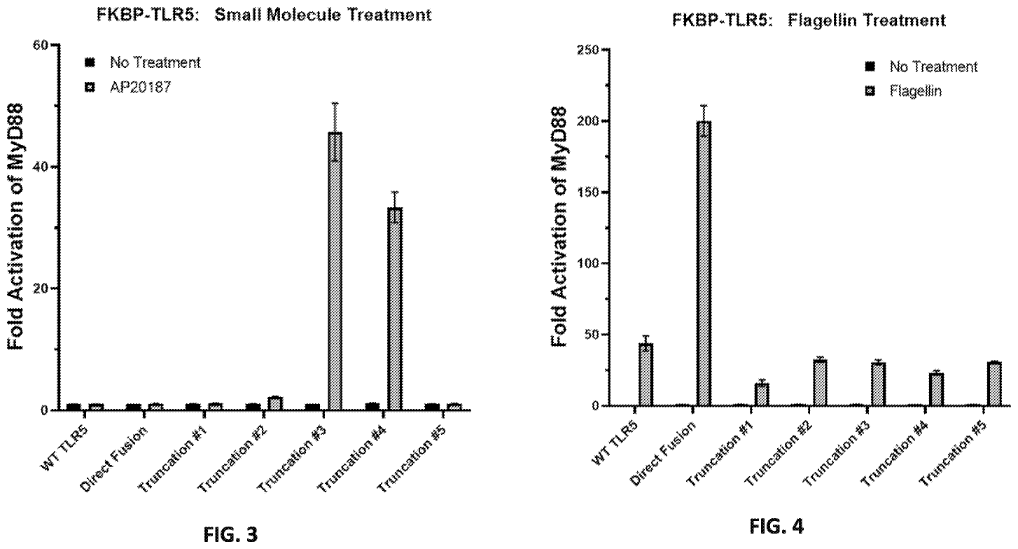

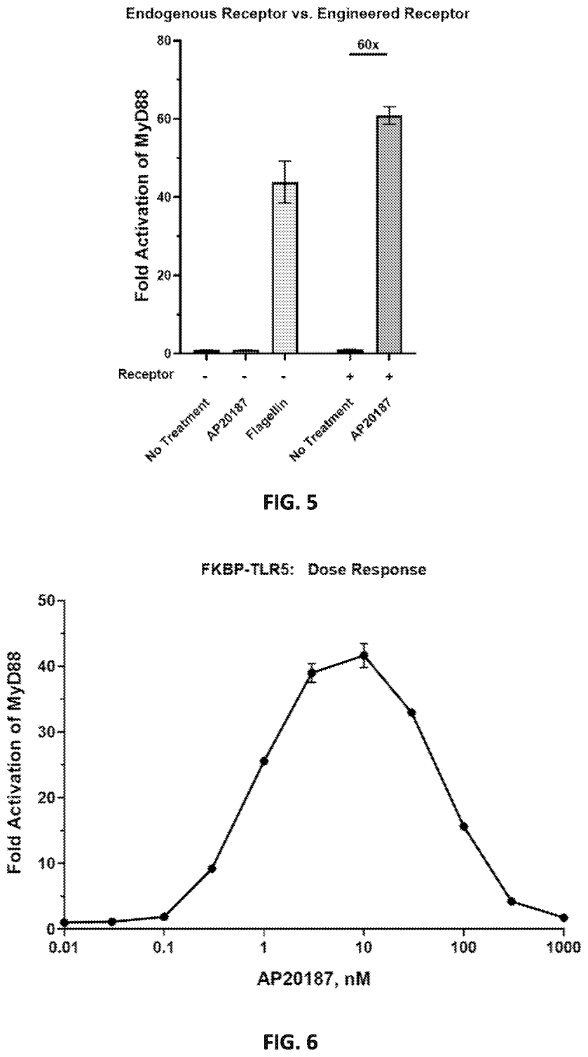

To determine where to truncate the TLR5 ECD such that the binding domain could be redirected to expand the soluble factor repertoire, the ECD was also methodically truncated at specific locations prior to fusing to FKBP. Results indicated that truncated FKBP-TLR5 receptors can be activated with AP20187 but not by the endogenous TLR5 ligand flagellin. The graph of FIG. 3 plots the Nκβ activity for Jurkat-NFKκβ-GFP reporter cells engineered with or without truncated receptor constructs and treated with AP20187 activator for 8-24 hr. The graph of FIG. 4 plots the NFκβ activity for Jurkat-NFκβ-GFP reporter cells engineered with or without truncated receptor constructs and treated with Flagellin activator for 8-24 hr. The graph of FIG. 5 plots NFκβ activity for Jurkat-NFκβ-GFP reporter cells engineered with or without receptor and treated with or without AP20187 or Flagellin activator for 8-24 hr. The graph of FIG. 6 plots NFκβ activity for receptor-engineered Jurkat-NFκβ-GFP reporter cells treated with a 0.01-1000 nM dose range of AP20187 activator for 8-24 hr. Together these results demonstrate that the provided receptor can be engineered to activate by a custom ligand and to be orthogonal to endogenous input signals.

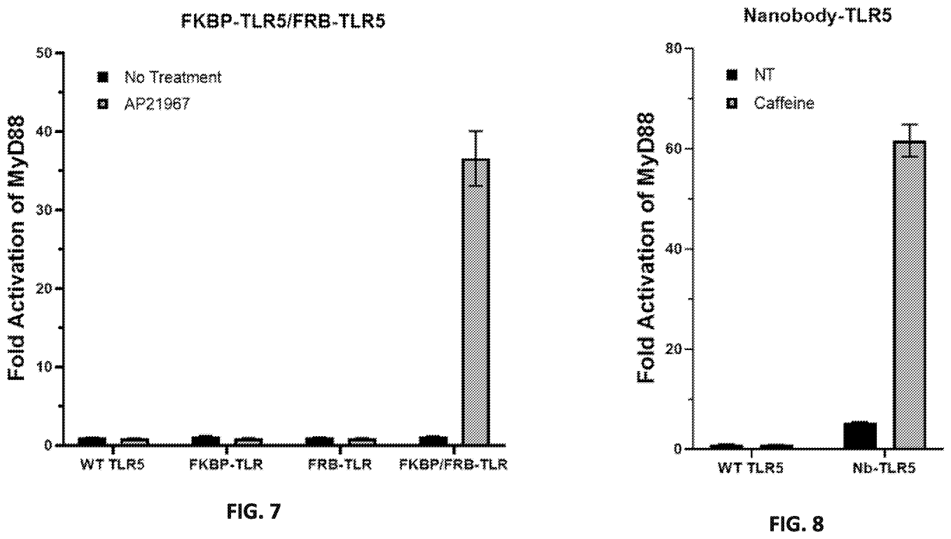

The receptor was further modified to activate upon heterodimerization (A/B system) by dimerizing two different receptor constructs. FKBP heterodimerizes with FKBP-rapamycin-binding (FRB, protein fragment of human mTOR) with the addition of AP21967 (protein-protein interaction inducing macrocyclic small molecule), and an FKBP-TLR5/FRB-TLR5 model receptor system was engineered and activated with AP21967. The graph of FIG. 7 plots NFκβ activity for receptor-engineered Jurkat-NFκβ-GFP reporter cells treated with or without AP21967 activator for 8-24 hr, demonstrating intramembrane signal pathway activation by the heterodimerization system.

The receptor ECD was also expanded beyond endogenous proteins to engineered nanobodies that bind to small molecules. For example, FIG. 8 presents results for a nanobody-fused chimeric transmembrane receptor polypeptide (Nb-TLR5) engineered and activated by treatment with the asymmetric monomeric small molecule, caffeine. The graph of FIG. 8 plots NFκβ activity for receptor-engineered Jurkat-NFκβ-GFP reporter cells treated with or without caffeine activator for 8-24 hr. This data further shows that the provided receptor can be engineered to any soluble factor input utilizing a binding domain that can recognize a soluble factor through either a homo-oligomerization or hetero-oligomerization activation mode.

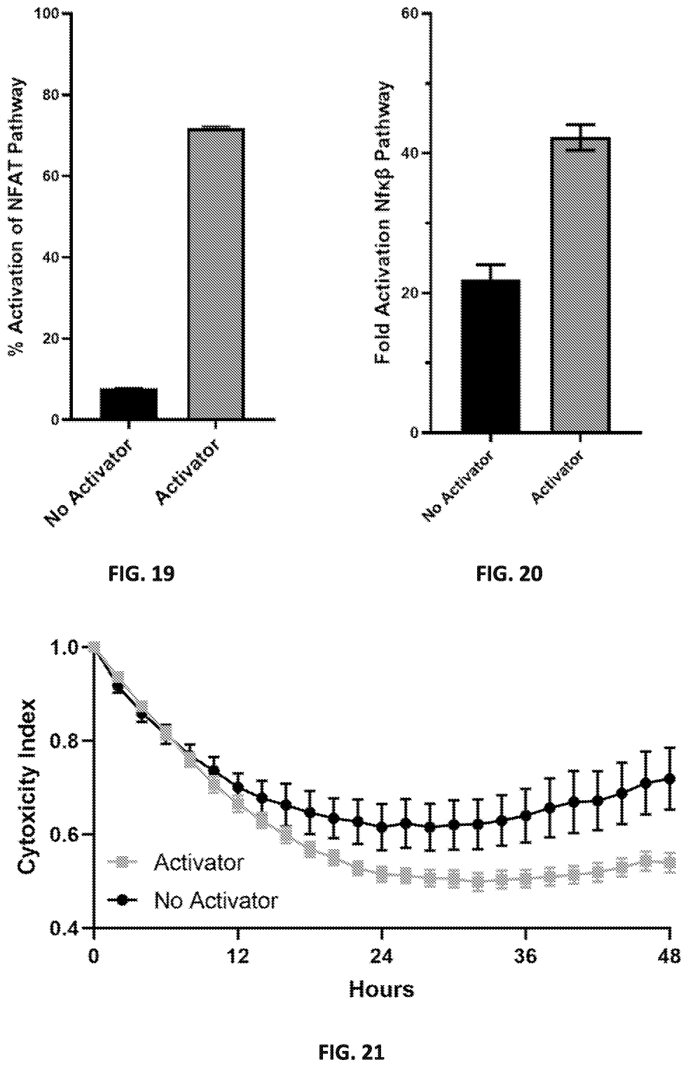

The ability of the provided chimeric transmembrane receptor polypeptides to recognize ligands including multimeric small molecules was also demonstrated. For example, FIG. 10 presents results for receptor recognition of different activators that were each a custom homodimer of a synthetic Mcl-1 molecular glue small molecule. The homodimers included pegylated linkers of length PEG1 (Activator 1), PEG3 (Activator 2), and PEG7 (Activator 3). The graph of FIG. 10 plots NFκβ activity for receptor-engineered Jurkat-NFκβ-GFP reporter cells treated with or without the dimeric Mcl-1 small molecule activators of different pegylated linker lengths for 8-24 hr. Further, FIGS. 19 and 20 present results for receptor recognition of an activator that was a tetramer of biotin. The graph of FIG. 19 plots NFAT activity for receptor-engineered Jurkat-NFAT-GFP reporter cells treated with or without the tetrameric biotin small molecule activator for 8-24 hr. The graph of FIG. 20 plots NFκβ activity for receptor-engineered Jurkat-NFκβ-GFP reporter cells treated with or without the tetrameric biotin small molecule activator for 8-24 hr.

### Example 2

### Extracellular Engineering of Receptor Polypeptides Recognizing Peptide Activators

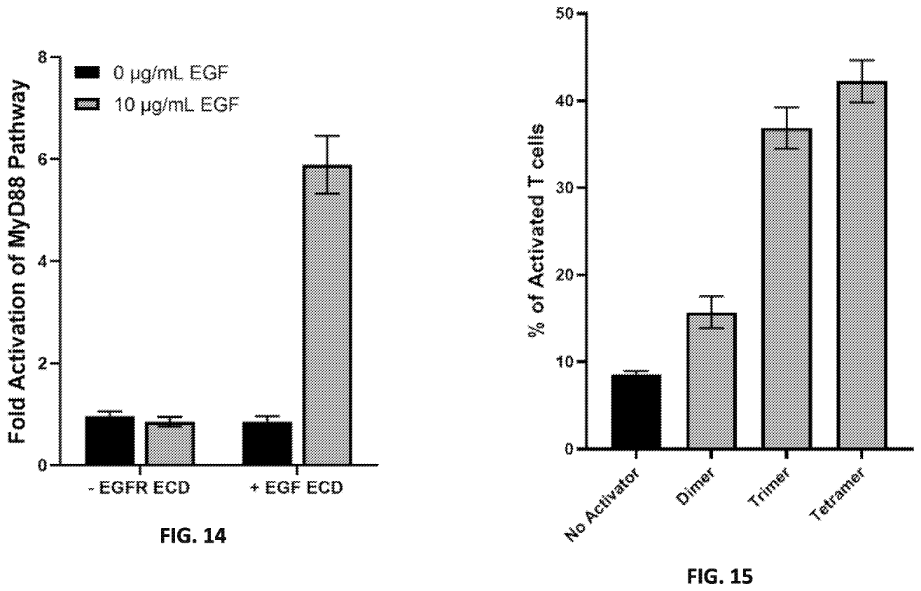

The ability of the provided chimeric transmembrane receptor polypeptides to recognize ligands including peptides or proteins was also demonstrated. For example, FIG. 14 presents results for receptor recognition of the natural epidermal growth factor (EGF) protein. The graph of FIG. 14 plots NFκβ activity for receptor-engineered Jurkat-NFκβ-GFP reporter cells treated with or without EGF activator for 8-24 hr, demonstrating activation of intramembrane signal pathways selectively in the presence of the peptide ligand.

### Example 3

### Extracellular Engineering of Receptor Polypeptides Recognizing Oligonucleotide Activators

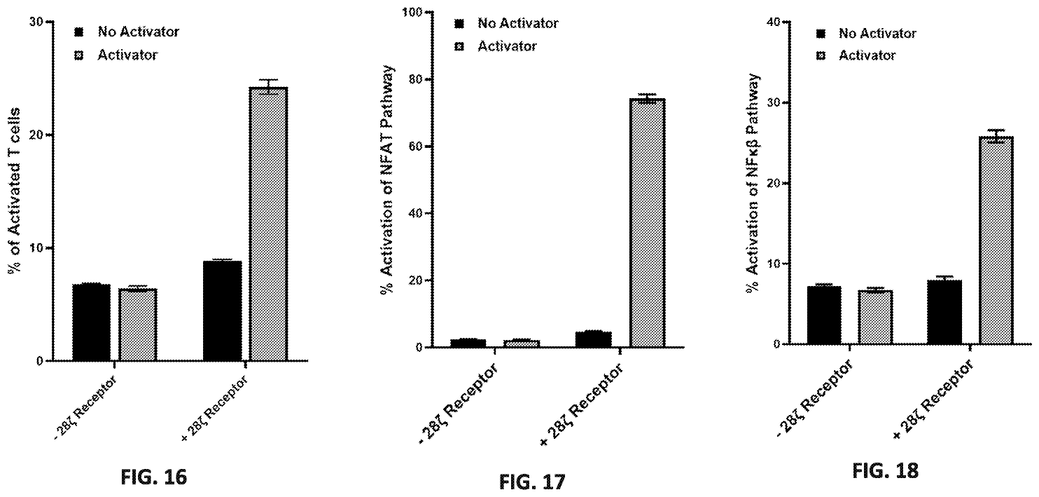

The ability of the provided chimeric transmembrane receptor polypeptides to recognize ligands including oligonucleotides was also demonstrated. For example, FIG. 16 presents results for receptor recognition of a trimeric DNA origami structure including an oligonucleotide sequence linked to a double stranded DNA scaffold linker. The graph of FIG. 16 plots NFκβ activity for receptor-engineered Jurkat-NFκβ-GFP reporter cells treated with or without the trimeric oligonucleotide activator for 8-24 hr, demonstrating activation of intramembrane signal pathways selectively in the presence of the oligonucleotide ligand. Also, FIG. 15 presents results for receptor recognition of a biotin-conjugated oligonucleotide (DNA origami) activator containing 2, 3, or 4 biotin molecules. The graph of FIG. 15 plots CD69 surface expression levels for receptor-engineered Jurkat cells treated with or without the dimeric, trimeric, or tetrameric biotin-conjugated oligonucleotide ligand for 8-24 hr.

### Example 4

### Receptor Expression and Localization

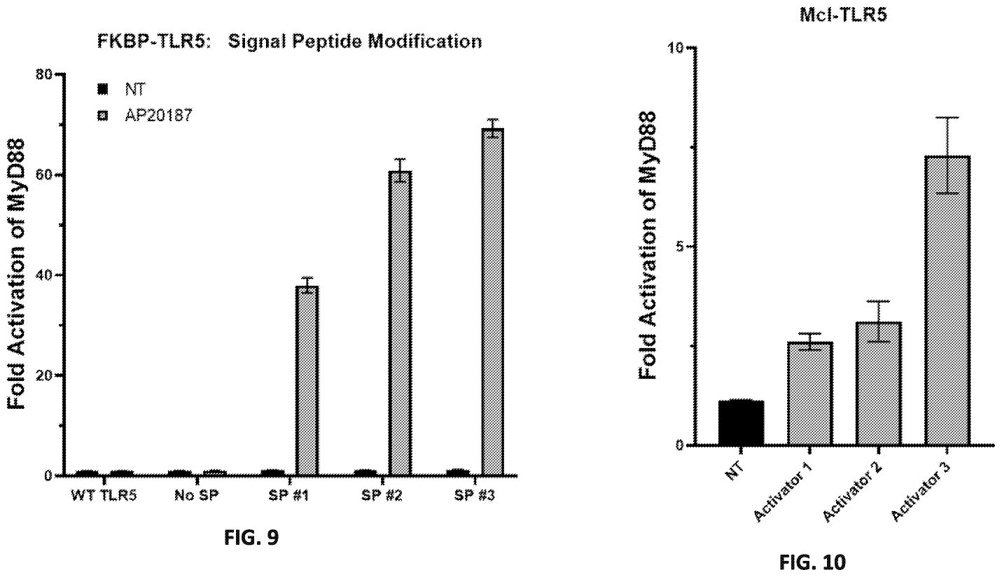

The expression and localization of the provided chimeric transmembrane receptor polypeptide has been engineered via the signal peptide (SP) and transmembrane domain (TMD). The SP affects the ability a host cell to process and traffic the receptor to, for example, the cellular membrane. The choice of SP has an effect on the activation potential of the FKBP-TLR5 receptor, as shown in FIG. 9. The graph of FIG. 9 plots NFκβ activity for receptor-engineered Jurkat-NFκβ-GFP reporter cells with three different signal peptides, where the cells were treated with or without AP20187 activator for 8-24 hr.

Domain swapping of the TMD can also be used to control cellular localization. For example, the TLR5 TMD has been swapped for the endosomal TLR7 TMD to redirect the receptor from being a cell membrane bound (TLR5) receptor to an endosomal bound (TLR7) receptor. Such successful receptor localization control allows for sampling of the endosome and exosome space with custom signal output, which has applications in immune cells, e.g., macrophages.

### Example 5

### Intracellular Domain Engineering of Receptor Polypeptides within the TLR Family

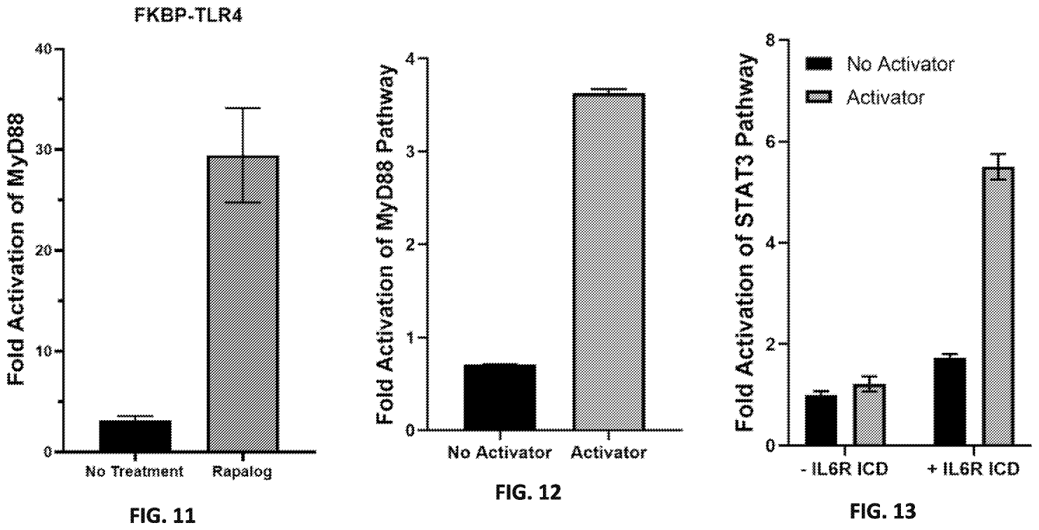

The suitability of TLR family receptor domains other than those of TLR5 for use with the provided chimeric transmembrane receptor polypeptides has also been demonstrated. For example, FIG. 11 presents results for a chimeric transmembrane receptor polypeptide constructed by linking FKBP to domains from TLR4. The graph plots NFκβ activity for the receptor-engineered Jurkat-NFκβ-GFP reporter cells treated with or without AP20187 activator for 8-24 h, showing that the TLR4-based receptor can activate intramembrane signal pathways similarly to the TLR5-based receptor of FIG. 2.

The suitability of intracellular domain swapping within a receptor family is shown in FIG. 12. The graph of FIG. 12 presents the results of a transmembrane receptor polypeptide that was constructed by linking the FKBP extracellular domain to the TLR5 transmembrane domain to the TLR4 intracellular domain. The graph plots NFκβ activity for the receptor-engineered Jurkat-NFκβ-GFP reporter cells treated with or without AP20187 activator for 8-24 hr, demonstrating that the TLR4/TLR5 chimera can activate intramembrane signaling pathways.

### Example 6

### Intracellular Domain Engineering of Receptor Polypeptides Outside the TLR Family

The provided chimeric transmembrane receptor polypeptides can activate signaling pathways through new intracellular domains beyond the TLR family. FIG. 13 presents results for a transmembrane receptor polypeptide containing an FKBP extracellular domain, TLR4 transmembrane domain, and an Interleukin-6 type I cytokine receptor (IL6R) intracellular domain. The graph plots STAT3 activity of receptor-engineered HEK293T-STAT3-GFP reporter cells treated with or without AP20187 activator for 8-24 hr, demonstrating that the receptor architecture can activate this signaling pathway. FIGS. 17 and 18 present NFκβ and NFAT activity levels for intracellular domain swapping with the immunoglobulin superfamily (IgSF) domains CD3ζ and CD28. FIG. 20 presents results for intracellular domain swapping with a tumor necrosis factor (TNF) family domain 4-1BB (CD137).

### Example 7

### Receptor Engineering of Natural Killer Cells

While the preceding examples each involved the engineering of T cells from the Jurkat cancer cell line, the provided chimeric transmembrane receptor polypeptides can be used to engineer a variety of host cell types as discussed above. For example, FIG. 21 presents results for the engineering of natural killer cells to improve their ability to kill MHC-cells. The graph of FIG. 21 plots the cytotoxic index of the engineered NK-92 cells as measured by live cell imaging, demonstrating the selective activation of improved cell killing in response to the presence of the activator for the FKBP-TLR5 chimeric transmembrane receptor polypeptide introduced to the engineered cells.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the disclosure. In addition, each reference provided herein is incorporated by reference in its entirety for all purposes to the same extent as if each reference was individually incorporated by reference.

What is claimed is:

1. A chimeric transmembrane receptor polypeptide configured to oligomerize upon recognition of an extramembrane signal by the chimeric transmembrane receptor polypeptide, the chimeric transmembrane receptor polypeptide comprising: 3
an extramembrane domain;
a transmembrane domain; and
an intramembrane domain, wherein the intramembrane domain is configured to induce activation of one or more intramembrane signal pathways upon oligomerization of the chimeric transmembrane receptor polypeptide.

2. The chimeric transmembrane receptor polypeptide of claim 1, wherein the chimeric transmembrane receptor polypeptide is configured to dimerize upon recognition of the extramembrane signal, and wherein the intramembrane domain is configured to induce activation of the one or more intramembrane signal pathways upon dimerization of the chimeric transmembrane receptor polypeptide.

3. The chimeric transmembrane receptor polypeptide of claim 1, wherein the extramembrane domain comprises an FK506 binding protein (FKBP) family domain, a bromodomain and extra terminal domain (BET) family domain, a gibberellin-insensitive dwarf (GID) family domain, a B-cell lymphoma 2 (Bcl-2) family domain, or a variant or fragment thereof.

4. The chimeric transmembrane receptor polypeptide of claim 1, wherein the extramembrane domain comprises a soluble N-ethylmaleimide-sensitive factor (NSF) attachment protein (SNAP) receptor domain, a biotin receptor domain, an epidermal growth factor receptor domain, an estrogen receptor domain, an androgen receptor domain, an insulin receptor domain, a programmed cell death protein-1 (PD-1) domain, an AXL receptor tyrosine kinase domain, a single-chain variable fragment (scFv) domain, a nanobody domain, or a variant or fragment thereof.

5. The chimeric transmembrane receptor polypeptide of claim 1, wherein the extramembrane domain comprises a toll-like receptor (TLR) family domain, an ErbB receptor family domain, a type I cytokine receptor family domain, a type II cytokine receptor family domain, a transforming growth factor beta (TGFβ) receptor family domain, a tumor necrosis factor (TNF) receptor family domain, an immunoglobulin superfamily (IgSF) domain, a tropomyosin receptor kinase (trk) family domain, a glial cell-derived neurotrophic factor (GDNF) receptor family domain, or a variant or fragment thereof.

6. The chimeric transmembrane receptor polypeptide of claim 1, wherein the transmembrane domain comprises a TLR family domain, an ErbB receptor family domain, a type I cytokine receptor family domain, a type II cytokine receptor family domain, a TGFβ receptor family domain, a TNF receptor family domain, an IgSF domain, a trk family domain, a GDNF receptor family domain, or a variant or fragment thereof.

7. The chimeric transmembrane receptor polypeptide of claim 1, wherein the intramembrane domain comprises a TLR family domain, an ErbB receptor family domain, a type I cytokine receptor family domain, a type II cytokine receptor family domain, a TGFβ receptor family domain, a TNF receptor family domain, an IgSF domain, a trk family domain, a GDNF receptor family domain, a gene editing nuclease domain, a transcriptional controller domain, an RNA controller domain, a protein controller domain, or a variant or fragment thereof.

8. The chimeric transmembrane receptor polypeptide of claim 1, further comprising:
one or more additional extramembrane domains.

9. The chimeric transmembrane receptor polypeptide of claim 1, further comprising:
one or more additional intramembrane domains.

10. The chimeric transmembrane receptor polypeptide of claim 1, further comprising:
a signal peptide.

11. The chimeric transmembrane receptor polypeptide of claim 10, wherein the signal peptide comprises a TLR family signal peptide, a CD3ε signal peptide, a CD8α signal peptide, an IgK signal peptide, an IgL signal peptide, a mouse CD4 signal peptide, or a variant or fragment thereof.

12. The chimeric transmembrane receptor polypeptide of claim 10, wherein the signal peptide comprises an oligomerization peptide, a pro-clustering peptide, an anti-clustering peptide, or a variant or fragment thereof.

13. The chimeric transmembrane receptor polypeptide of claim 1, further comprising:
one or more linker peptide sequences.

14. The chimeric transmembrane receptor polypeptide of claim 13, wherein the one or more linker peptide sequences comprise a GGS linker sequence, a GGSGGSGGS linker sequence, a GS linker sequence, a GSGSGS linker sequence, an ESKYGPPAPPAP (mutant IgG4 hinge) linker sequence, an ESKYGPPCPPCP (IgG4 hinge), or a combination thereof.

15. The chimeric transmembrane receptor polypeptide of claim 13, wherein the one or more linker peptide sequences include an oligomerization peptide sequence, a pro-clustering peptide sequence, an anti-clustering peptide sequence, or a variant or fragment thereof.

16. The chimeric transmembrane receptor polypeptide of claim 1, wherein the extramembrane signal comprises a ligand capable of binding to the extramembrane domain.

17. The chimeric transmembrane receptor polypeptide of claim 16, wherein the ligand comprises a small molecule.

18. The chimeric transmembrane receptor polypeptide of claim 16, wherein the ligand comprises an oligonucleotide.

19. The chimeric transmembrane receptor polypeptide of claim 16, wherein the ligand comprises a peptide, a protein, a polysaccharide, a lipid, or a combination thereof.

20. The chimeric transmembrane receptor polypeptide of claim 16, wherein the ligand comprises an antibody, a nanobody, or an scFv.

21. The chimeric transmembrane receptor polypeptide of claim 16, wherein the ligand comprises a metabolite.

22. The chimeric transmembrane receptor polypeptide of claim 1, wherein the extramembrane signal comprises a change in temperature or pH.

23. The chimeric transmembrane receptor polypeptide of claim 1, wherein the extramembrane signal comprises a change in sound or electromagnetic radiation.

24. The chimeric transmembrane receptor polypeptide of claim 1, wherein the extramembrane signal comprises a change in mechanical force.

25. The chimeric transmembrane receptor polypeptide of claim 1, wherein at least one of the one or more intramembrane signal pathways is an exogenous pathway.

26. The chimeric transmembrane receptor polypeptide of claim 1, wherein at least one of the one or more intramembrane signal pathways is a synthetic pathway.

27. The chimeric transmembrane receptor polypeptide of claim 1, wherein the one or more intramembrane signal pathways comprise genome sequence editing. transcription activation or repression, epigenetic modifications, genome translocation and rearrangement, RNA expression or degradation, RNA splicing or processing, post-transcription modifications of mRNA or mRNA, post-translational modifications of proteins, cleavage or proteolysis of proteins, production or degradation of metabolites or other chemistries, trafficking of signaling molecules, cell cycle control, cell differentiation or reprogramming, T cell activation or exhaustion, programmed cell death, cell trafficking, secretion of cytokines or hormones, neuronal activity, macrophage phagocytosis, neutrophil NETpoptosis, immunological synapse formation, myeloid cell degranulation, antigen presentation, secretion or hypermutation of antibodies, production of oncolytic virus, or a combination thereof.

28. A system comprising:
a membrane separating an extramembrane region from an intramembrane region;
the chimeric transmembrane receptor polypeptide of claim 1, wherein the extramembrane domain is located within the extramembrane region, and wherein the intramembrane domain is located within the intramembrane region. 6

29. The system of claim 28, wherein the membrane is a cellular membrane, a nuclear membrane, an organelle membrane, or a vesicle membrane.

30. The system of claim 28, further comprising:
a trans-acting receptor polypeptide.

31. The system of claim 30, wherein the trans-acting receptor polypeptide is a chimeric antigen receptor (CAR), a T cell antigen receptor (TCR), a Synthetic Notch (SynNotch) receptor, a GPCR TANGO receptor, a CRISPR ChaCha receptor, a B cell receptor (BCR), C-type lectin-like receptor Ly49, a CD94-NKG2C/E/H heterodimeric receptor, an NKG2D receptor, a DNAM-1/CD226 nectin/nectin-like binding receptor, a CRTAM nectin/netin-like binding receptor, a member of the natural cytotoxicity receptor (NCR) family, a CD64 Fc receptor, a CD32 Fc receptor, a CD16a Fc receptor, a CD16b Fc receptor, a CD23 Fc receptor, a CD89 Fc receptor, a CD351 Fc receptor, an FcεRI Fc receptor, or an FcRn Fc receptor.

32. A host cell comprising the chimeric transmembrane receptor polypeptide of claim I or the system of claim 28.

33. The host cell of claim 32, wherein the host cell expresses the chimeric transmembrane receptor polypeptide.

34. The host cell of claim 32, wherein the host cell is a lymphocyte, a phagocytic cell, a granulocytic cell. or a dendritic cell.

35. The host cell of claim 34, wherein the lymphocyte is a T cell, a B cell, a natural killer (NK) cell, or an innate lymphoid cell (ILC).

36. The host cell of claim 35, wherein the T cell is a CD4+ helper αβT cell, a CD8⁺ killer αβT cell, a δγT cell, or a natural killer T (NKT) cell.

37. The host cell of claim 34, wherein the phagocytic cell is a monocyte or a macrophage.

38. The host cell of claim 34, wherein the granulocytic cell is a neutrophil, a basophil, an eosinophil, or a mast cell.

39. The host cell of claim 32, wherein the host cell is a stem cell or a progenitor cell.

40. The host cell of claim 39, wherein the host cell is an induced pluripotent stem cell (iPSC), an embryonic stem cell (ESC), an adult stem cell, or a mesenchymal stem cell (MSC).

41. The host cell of claim 39, wherein the progenitor cell is a neural progenitor cell. a skeletal progenitor cell, a muscle progenitor cell, a fat progenitor cell, a heart progenitor cell, a chondrocyte, or a pancreatic progenitor cell.

42. A population of host cells, wherein each host cell of the population independently comprises the chimeric transmembrane receptor polypeptide of claim I or the system of claim 28.

43. A method for activating an intramembrane signal pathway, the method comprising providing the chimeric transmembrane receptor polypeptide of claim 1.

44. The method of claim 43, further comprising exposing the chimeric transmembrane receptor to the extramembrane signal.

45. A method for preventing or treating a disease in a subject, the method comprising administering to the subject an amount of the chimeric transmembrane receptor polypeptide of claim 1.

46. The method of claim 45, further comprising, subsequent to the administering, exposing the chimeric transmembrane receptor to the extramembrane signal.

47. The method of claim 46, wherein the exposing comprises introducing to the subject a therapeutically effective amount of the extramembrane signal.

48. The method of claim 45, wherein the disease is a cancer.

49. The method of claim 45, wherein the disease is a cancerous tumor.

50. The method of claim 49, wherein the cancerous tumor is a solid cancerous tumor.

51. The method of claim 49, wherein the cancerous tumor is a liquid cancerous tumor.

52. The method of claim 45, wherein the disease is an infectious disease.

53. The method of claim 52, wherein the infectious disease is a viral infectious disease or a bacterial infectious disease.

54. The method of claim 45, wherein the disease is an autoimmune disease.

55. The method of claim 45, wherein the disease is an age-related disease.

56. A method for healing a wound in a subject, the method comprising administering to the subject an amount of the chimeric transmembrane receptor polypeptide of claim 1.

57. The method of claim 56, further comprising, subsequent to the administering, exposing the chimeric transmembrane receptor to the extramembrane signal.

58. The method, of claim 57, wherein the exposing comprises introducing to the subject a therapeutically effective amount of the extramembrane signal.
